(19) United States
(12) Patent Application Publication
(10) Pub. No.: US 2010/0168077 A1
(43) Pub. Date: Jul. 1, 2010

(54) Novel Pyridine Derivatives, Processes for Preparing Them, Pharmaceutical Compositions Thereof

(75) Inventor: Duncan Hannah, Suffolk (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Brusells (BE)

(21) Appl. No.: 12/594,313

(22) PCT Filed: Mar. 31, 2008
(86) PCT No.: PCT/EP08/02525

§ 371 Date: Feb. 8, 2010

(30) Foreign Application Priority Data
Apr. 4, 2007 (GB) 0706649.1
May 19, 2007 (EP) 07010008.6

(51) Int. Cl. A61K 31/4427; C07D 401/14; A61K 31/496; C07D 401/04; A61K 31/4545; A61K 31/4439; A61P 11/00; A61P 11/06; A61P 37/08; A61P 11/02; A61P 15/00; A61P 13/10; A61P 13/08; A61P 17/06; A61P 9/10; A61P 13/12; A61P 1/04; A61P 25/28; A61P 19/02; A61P 35/00

(52) U.S. Cl. 514/210.2; 544/364; 514/253.01; 544/360; 546/279.1; 546/268.1; 546/193; 514/318; 514/343; 514/337

(57) ABSTRACT

The present invention concerns 2-amino pyridine derivatives of formula 1 processes for preparing them, pharmaceutical compositions containing them and their use as pharmaceuticals.

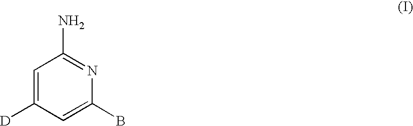

The present invention concerns novel 2-amino pyridine derivatives, processes for preparing them, pharmaceutical compositions containing them and their use as pharmaceuticals.

### BACKGROUND

To date a number of inflammatory actions of the H₄-receptor have been described: in vitro actions, calcium mobilisation and chemotaxis of murine mast cells (Hofstra et al. 2003) and eosinophils (Buckland et al., 2003; Ling et al., 2004), upregulation of adhesion molecules, CD11b/CD18 (Mac1) and CD54 on eosinophils (Buckland et al. 2003; Ling et al. 2004) and reduction in pro-inflammatory cytokine profiles following toll-like receptor (TLR) ligand stimulation of dendritic cells (Dunford et al. 2006); in vivo actions, histamine-induced mast cell recruitment (Thurmond et al., 2004), neutrophil infiltration in a mouse zymosan-induced peritonitis model (Thurmond et al. 2004) and zymosan-induced neutrophilia to the pleural cavity (Takeshita et al. 2003), eosinophil recruitment (Dunford et al. 2006; Douglas et al., 2006) and mediating itch/pruritis (Bell et al. 2004).

On this basis histamine H₄-receptor antagonists and inverse agonists may be used for the prophylaxis and treatment of different kind of diseases and disorders such as: respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal disfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

Patent application GB 2 071 092 discloses inner salts of triazone, pyrimidine and pyridinium derivatives, which are stated to have utility in the treatment of hypertension, congestive heart failure, Raynaud's disease, gangrene and other peripheral vascular diseases. Pyridines of these derivatives are substituted by N-oxide group or sulfoxide group.

Patent application EP 1 505 064 and WO 2005/054239 disclose 2-aminopyrimidine derivatives, useful for treatment of diseases associated with histamine H4 receptor activity.

Patent application WO 2004/024711 discloses substituted pyridine derivatives (4-amino pyridine derivatives) as anti-tumour agent.

It has now surprisingly been found, that some novel 2-amino-pyridine derivatives demonstrate therapeutic properties in this field.

In one aspect, the invention provides a compound having formula I or pharmaceutically acceptable salts thereof or stereoisomeric forms thereof, and the geometrical isomers, enantiomers, diastereoisomers, and pharmaceutically acceptable salts thereof

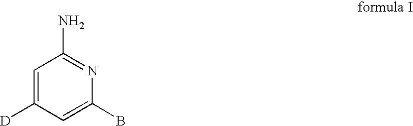

* represents the point of attachment to the rest of the molecule
wherein:

B is aryl optionally substituted with a substituent selected from the group consisting of C₁₋₆ alkyl, halogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member cycloalkyl, 3-10 member heterocycloalkyl, heteroaryl;

or B is a heteroaryl ring optionally substituted with a substituent selected from the group consisting of C₁₋₆ alkyl, halogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member cycloalkyl, 3-10 member heterocycloalkyl, heteroaryl;

or B is C₁₋₆ alkyl optionally substituted with a substituent selected from the group consisting of aryl, heteroaryl, 3-10 member cycloalkyl, 3-10 member heterocycloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine;

or B is C₂₋₆ alkenyl optionally substituted with a substituent selected from the group consisting of C₁₋₆ alkyl, aryl, heteroaryl, 3-10 member cycloalkyl, 3-10 member heterocycloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine;

or B is C₃₋₁₀ cycloalkyl optionally substituted by C₁₋₆ alkyl;

or B is C₃₋₁₀ cycloalkenyl optionally substituted by C₁₋₆ alkyl;

or B is a group of formula II

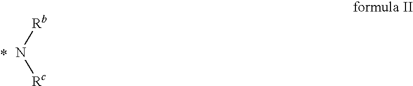

wherein

R^{b} is hydrogen or C₁₋₆ alkyl;
R^{c} is C₃₋₁₀ cycloalkyl optionally substituted by C₁₋₃ alkyl;
or R^{b} and R^{c} can form together with the nitrogen, a 3-10 member heterocycloalkyl or a heteroaryl optionally substituted by C₁₋₆ alkyl;
D is a group of formula III

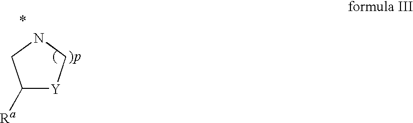

wherein

R^{a} is hydrogen or unsubstituted C₁₋₃ alkyl;
p is 1 or 2 or 3;
Y is a group of formula IV

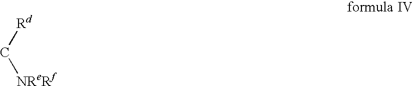

wherein

R^{d} is hydrogen or unsubstituted C₁₋₃ alkyl;
R^{e} is hydrogen or unsubstituted C₁₋₃ alkyl;
R^{f} is hydrogen or unsubstituted C₁₋₃ alkyl;
or Y is a group of formula V

N—R^{g} formula V

wherein

R^{g} is hydrogen or unsubstituted C₁₋₃ alkyl;
or D is a group of formula VI

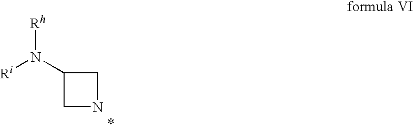

wherein

R^{h} is hydrogen or unsubstituted C₁₋₃ alkyl;
Rⁱ is hydrogen or unsubstituted C₁₋₃ alkyl;
or D is a group of formula VII

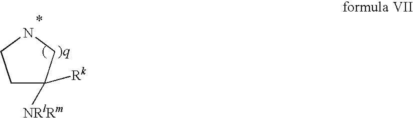

wherein

q is 1 or 2 or 3;
R^{k} is hydrogen or unsubstituted C₁₋₃ alkyl;
R^{l} is hydrogen or unsubstituted C₁₋₃ alkyl;
R^{m} is hydrogen or unsubstituted C₁₋₃ alkyl;
or D is group of formula VIII

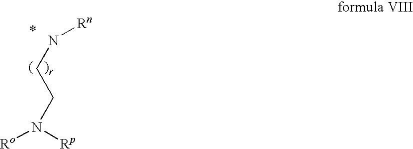

wherein

r is 1 or 2;
Rⁿ is hydrogen or unsubstituted C₁₋₃ alkyl;
R^{o} is hydrogen or unsubstituted C₁₋₃ alkyl;
R^{p} is hydrogen or unsubstituted C₁₋₃ alkyl;
or D is a group of formula IX

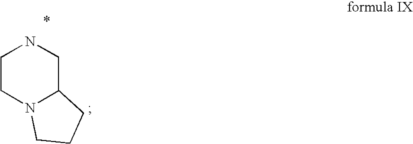

or D is a group of formula X

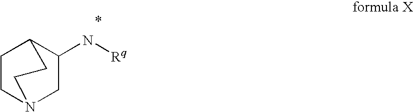

wherein

R^{q} is hydrogen or unsubstituted C₁₋₃ alkyl;
or D is a group of formula XI

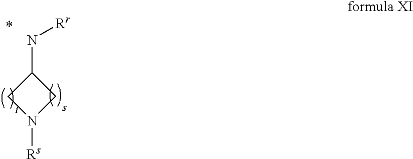

wherein

R^{r} is hydrogen or unsubstituted C₁₋₃ alkyl;
R^{s} is hydrogen or unsubstituted C₁₋₃ alkyl;
s is 1 or 2;
t is 1 or 2;
or D is a group of formula XII

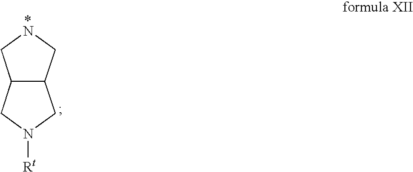

wherein

R^{t} is hydrogen or unsubstituted C₁₋₃ alkyl.

The term “alkyl”, as used herein, refers to saturated, monovalent or divalent hydrocarbon radicals having linear or branched moieties and containing 1-6 carbon atoms.

Alkyl groups may be optionally substituted by one or more groups selected from halogen, hydroxyl, C₁₋₃ alkoxy, amide, amino, ester, sulfonamide, sulfonic acid, aryl, heteroaryl. Usually alkyl is methyl.

The term “halogen”, as used herein, refers to an atom of chlorine, bromine, fluorine, iodine. Usually halogen is chlorine.

The term “alkenyl” as used herein, refers to a monovalent or divalent group, linear or branched, and containing 2 to 6 carbon atoms, derived from a saturated C₂₋₆ alkyl, as described above, having one double bond. Alkenyl groups can be optionally substituted by one or more groups selected from C₁₋₆ alkyl, aryl, heteroaryl, 3-10 member cycloalkyl, 3-10 member heterocycloalkyl, hydroxyl, C₁₋₃ alkoxy, haloalkoxy, carboxylic acid, ester, C₁₋₃ alkylsulfonyl, amide, sulfonamide, C₁₋₃ dialkylamine.

The term “C₃₋₁₀ cycloalkyl”, as used herein, refers to a monovalent or divalent group of 3 to 10 carbon atoms, derived from a saturated cyclic hydrocarbon. Cycloalkyl groups can be optionally substituted by one or more C₁₋₃ alkyl groups. Cycloalkyl groups can be monocyclic or polycyclic.

The term C₁₋₃ “alkoxy”, as used herein, refers to a group of formula —OR¹ wherein R¹ is an alkyl as defined above, containing 1 to 3 carbon atoms. Usually C₁₋₃ alkoxy group is methoxy.

The term C₁₋₃ “haloalkoxy”, as used herein, refers to a C₁₋₃ alkyl group, as defined above, substituted by 1 to 3 halogens. Usually haloalkoxy group is trifluoromethoxy.

The term “carboxylic acid” as used herein, refers to a group of formula —CO₂H.

The term “ester” as used herein, refers to a group of formula —CO₂R², wherein R² is a C₁₋₃ alkyl, group, as defined above.

The term “sulfonamide” as used herein, refers to a group of formula —SO₂NR³R⁴, wherein R³ is a C₁₋₃ alkyl group, as defined above and R⁴ is a C₁₋₃ alkyl group, as defined above, or refers to a group of formula —NHSO₂R⁵, wherein R⁵ is a C₁₋₃ alkyl group, as defined above.

The term “sulfonic acid” as used herein, refers to a group of formula —SO₃H.

The term “hydroxyl”, as used herein, refers to a group of formula —OH.

The term “amino”, as used herein, refers to a group of formula —NH₂.

The term “C₁₋₃ alkylsulfonyl” as used herein, refers to a group of formula —SO₂R⁶, wherein R⁶ is a C₁₋₃ alkyl group, as defined above.

The term “amide” as used herein, refers to a group of formula —NR⁷COR⁸, wherein R⁷ is a C₁₋₃ alkyl group, as defined above and R⁸ is a C₁₋₃ alkyl group, as defined above, or refers to a group of formula —CONR⁹, R¹⁰, wherein R¹⁰ is a C₁₋₃ alkyl group, as defined above and R⁹ is a C₁₋₃ alkyl group, as defined above.

The term “C₁₋₃ dialkylamine”, as used herein, refers to a group of formula —NR¹¹R¹², wherein R¹¹ is a C₁₋₃ alkyl group, as defined above and R¹² is a C₁₋₃ alkyl group as defined above.

The term “3-10 member heterocycloalkyl”, as used herein refers to a 3 to 10 cycloalkyl member ring, as defined above, containing at least one heteroatom selected from O or N or S or combinations of at least two thereof, interrupting the carbocyclic ring structure. The heterocyclic ring can be interrupted by —C═O. The S heteroatom can be oxidized. Heterocycloalkyls can be monocyclic or polycyclic.

The term “C₁₋₃ haloalkyl”, as used herein, refers to a C₁₋₃ alkyl group, as defined above, substituted by 1 to 3 halogens. Usually the alkyl group is methyl and the halogen is fluoro. Usually “haloalkyl” group is trifluoromethyl.

The term “aryl” as used herein, refers to an organic moiety derived from an aromatic hydrocarbon consisting of a ring or multiple rings, containing 6 to 10 carbon atoms by removal of one hydrogen atom, which can optionally be substituted by one or more groups selected from C₁₋₆ alkyl, halogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl. Usually aryl is phenyl, 4-chlorophenyl, 3-methylphenyl.

The term “heteroaryl”, as used herein refers to an aryl ring, as described above, containing at least one heteroatom selected from O or N or S or combinations of at least two thereof, interrupting the carbocyclic ring structure. The heteroaryl ring can be interrupted by —C═O. The S heteroatom can be oxidized. Heteroaryls can optionally be substituted by one or more groups selected from C₁₋₆ alkyl, halogen, hydrogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamino, 3-10 member heterocycloalkyl heterocycloalkyl, heteroaryl.

The term “C₃₋₁₀ cycloalkenyl”, as used herein, refers to a monovalent or divalent group of 3 to 10 carbon atoms, derived from a saturated C₃₋₁₀ cycloalkyl, as described above, having one double bond. Cycloalkenyl groups can be monocyclic or polycyclic. Cycloalkenyl groups can be substituted by C₁₋₆ alkyl groups as defined above.

In one embodiment of the invention B is aryl optionally substituted with a substituent selected from the group consisting of C₁₋₆ alkyl, halogen, hydrogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl. Usually B in the present case is 4-chlorophenyl, 3 methyl-phenyl, 4-trifluoromethylphenyl, 4-trifluoromethoxyphenyl.

In another embodiment of the invention B is a heteroaryl ring optionally substituted with a substituent selected from the group consisting of C₁₋₆ alkyl, halogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl.

In another embodiment of the invention B is C₁₋₆ alkyl optionally substituted with a substituent selected from the group consisting of aryl, heteroaryl, 3-10 member cycloalkyl, 3-10 member heterocycloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine.

In another embodiment of the invention B is C₂₋₆ alkenyl optionally substituted with a substituent selected from the group consisting of C₁₋₆ alkyl, aryl, heteroaryl, 3-10 member cycloalkyl, 3-10 member heterocycloalkyl, hydroxy, C₁₋₃ alkoxy, C₁₋₃ haloalkoxy, carboxylic acid, ester, C₁₋₃ alkylsulfonyl, amide, sulfonamide, C₁₋₃ dialkylamine.

In another embodiment of the invention B is C₃₋₁₀ cycloalkyl optionally substituted by C₁₋₆ alkyl. Usually B in the present case is 4-methylcyclohex-1-en-1yl, 6-adamantan-2-yl.

In another embodiment of the invention B is C₃₋₁₀ cycloalkenyl optionally substituted by C₁₋₆ alkyl.

Usually B in the present case is 6-cyclohex-1-en-1-yl.

In another embodiment of the invention B is a group of formula II wherein R^{b} is hydrogen, C₁₋₆ alkyl; R^{c} is C₃₋₁₀ cycloalkyl optionally substituted by C₁₋₃ alkyl. Usually B in the present case is amino-cycloheptyl.

In another embodiment of the invention B is a group of formula II wherein R^{b} and R^{c} can form together with the nitrogen atom, a 3-10 member heterocycloalkyl or heteroaryl optionally substituted by C₁₋₆ alkyl. Usually B in the present case is 4-methylpiperazin-1-yl, 2-methylpyrrolidin-1-yl.

In one embodiment of the invention D is a group of formula III wherein R^{a} is C₁₋₃ alkyl, hydrogen; and p is 1 or 2 or 3; and Y is a group of formula IV wherein R^{d} is hydrogen or C₁₋₃ alkyl; and R^{e} is hydrogen or C₁₋₃ alkyl; and R^{f} is hydrogen or C₁₋₃ alkyl. Usually D in the present case is 3-methylaminopyrrolidin-1-yl, 3-aminopyrrolidin-1-yl, (3R)-3N-methylaminopyrrolidin-1-yl.

In another embodiment of the invention D is a group of formula III wherein R^{a} is C₁₋₃ alkyl, hydrogen; and p is 1, 2 or 3; and Y is a group of formula V wherein R^{g} is hydrogen or C₁₋₃ alkyl. Usually D in the present case is 4-methylpiperazin-1-yl.

In another embodiment of the invention D is a group of formula VI wherein R^{h} is hydrogen or C₁₋₃ alkyl; and Rⁱ is hydrogen or C₁₋₃ alkyl.

In another embodiment of the invention D is a group of formula VII wherein q is 1 or 2 or 3; and R^{k} is hydrogen or C₁₋₃ alkyl; and R^{l} is hydrogen or C₁₋₃ alkyl; and R^{m} is hydrogen or C₁₋₃ alkyl.

In another embodiment of the invention D is a group of formula VIII wherein r is 1 or 2; and Rⁿ is hydrogen or C₁₋₃ alkyl; and R^{o} is hydrogen or C₁₋₃ alkyl; and R^{p} is hydrogen or C₁₋₃ alkyl.

In another embodiment of the invention D is a group of formula IX.

In another embodiment of the invention D is a group of formula X wherein R^{q} is hydrogen or C₁₋₃ alkyl.

In another embodiment of the invention D is a group of formula XI wherein R^{r} is hydrogen or C₁₋₃ alkyl; and R^{s} is hydrogen or C₁₋₃ alkyl; and s is 1 or 2; and t is 1 or 2.

In another embodiment of the invention D is a group of formula XII wherein R^{t} is hydrogen or C₁₋₃ alkyl.

In one embodiment of the invention R^{a} is C₁₋₃ alkyl, hydrogen. Usually R^{a} is hydrogen.

In one embodiment of the invention R^{b} is hydrogen, C₁₋₆ alkyl or together with R^{c} and the nitrogen atom can form a 3-10 member heterocycloalkyl or a heteroaryl optionally substituted by C₁₋₆ alkyl. Usually R^{b} is hydrogen or together with R^{c} and the nitrogen atom 2-methylaminopyrrolidin-1-yl, 4-methylpiperidin-1-yl.

In one embodiment of the invention R^{c} is C₃₋₁₀ cycloalkyl optionally substituted by C₁₋₃ alkyl

or R^{b} and R^{c} can form together with the nitrogen atom a 3-10 member heterocycloalkyl or heteroaryl optionally substituted by C₁₋₆ alkyl. Usually R^{c} is cycloheptyl or together with R^{b} and the nitrogen atom forms 2-methylaminopyrrolidin-1-yl, 4-methylpiperidin-1-yl.

In one embodiment of the invention p is 1 or 2 or 3. Usually p is 1 or 2.

In one embodiment of the invention R^{d} is hydrogen or unsubstituted C₁₋₃ alkyl. Usually R^{d} is hydrogen.

In one embodiment of the invention R^{e} is hydrogen or unsubstituted C₁₋₃ alkyl. Usually R^{e} is hydrogen, methyl.

In one embodiment of the invention R^{f} is hydrogen or unsubstituted C₁₋₃ alkyl. Usually R^{f} is hydrogen, methyl.

In one embodiment of the invention R^{g} is hydrogen or unsubstituted C₁₋₃ alkyl. Usually R^{g} is methyl.

In one embodiment of the invention R^{h} is hydrogen or unsubstituted C₁₋₃ alkyl.

In one embodiment of the invention Rⁱ is hydrogen or unsubstituted C₁₋₃ alkyl.

In one embodiment of the invention q is 1 or 2 or 3.

In one embodiment of the invention R^{k} is hydrogen or unsubstituted C₁₋₃ alkyl.

In one embodiment of the invention R^{l} is hydrogen or unsubstituted C₁₋₃ alkyl.

In one embodiment of the invention R^{m} is hydrogen or unsubstituted C₁₋₃ alkyl.

In one embodiment of the invention r is 1 or 2.

In one embodiment of the invention Rⁿ is hydrogen or unsubstituted C₁₋₃ alkyl.

In one embodiment of the invention R^{o} is hydrogen or unsubstituted C₁₋₃ alkyl.

In one embodiment of the invention R^{p} is hydrogen or unsubstituted C₁₋₃ alkyl.

In one embodiment of the invention R^{q} is hydrogen or unsubstituted C₁₋₃ alkyl.

In one embodiment of the invention R^{r} is hydrogen or unsubstituted C₁₋₃ alkyl.

In one embodiment of the invention R^{s} is hydrogen or unsubstituted C₁₋₃ alkyl.

In one embodiment of the invention s is 1 or 2.

In one embodiment of the invention t is 1 or 2.

In one embodiment of the invention R^{t} is hydrogen or unsubstituted C₁₋₃ alkyl.

In one embodiment of the invention B is aryl optionally substituted with a substituent selected from the group consisting of C₁₋₆ alkyl, halogen, hydrogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl; and D is a group of formula III wherein R^{a} is C₁₋₃ alkyl, hydrogen; and p is 1 or 2 or 3; and Y is a group of formula IV wherein R^{d} is hydrogen or C₁₋₃ alkyl; R^{e} is hydrogen or C₁₋₃ alkyl; R^{f} is hydrogen or C₁₋₃ alkyl.

In another embodiment of the invention B is C₃₋₁₀ cycloalkyl optionally substituted by C₁₋₆ alkyl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

In another embodiment of the invention B is C₃₋₁₀ cycloalkenyl optionally substituted by C₁₋₆ alkyl; and D is a group of formula III wherein R^{a} is C₁₋₃ alkyl, hydrogen; and p is 1 or 2 or 3; and Y is a group of formula IV wherein R^{d} is hydrogen or C₁₋₃ alkyl; R^{e} is hydrogen or C₁₋₃ alkyl; R^{f} is hydrogen or C₁₋₃ alkyl.

In another embodiment of the invention B is C₃₋₁₀ cycloalkenyl optionally substituted by C₁₋₆ alkyl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

In another embodiment of the invention B is aryl optionally substituted with a substituent selected from the group consisting of C₁₋₆ alkyl, halogen, hydrogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl; and D is a group of formula III wherein R^{a} is C₁₋₃ alkyl, hydrogen; and p is for 2 or 3; and Y is a group of formula V wherein R^{g} is hydrogen or C₁₋₃ alkyl.

In another embodiment of the invention B is a group of formula II wherein R^{b} is hydrogen, C₁₋₆ alkyl; R^{c} is C₃₋₁₀ cycloalkyl optionally substituted by C₁₋₃ alkyl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

In another embodiment of the invention B is a group of formula II wherein R^{b} and R^{c} can form together with the nitrogen atom a 3-10 member heterocycloalkyl or heteroaryl optionally substituted by C₁₋₆ alkyl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

In another embodiment of the invention B is a group of formula II wherein R^{b} and R^{c} can form together with the nitrogen atom a 3-10 member heterocycloalkyl or heteroaryl optionally substituted by C₁₋₆ alkyl; and D is a group of formula III wherein R^{a} is C₁₋₃ alkyl, hydrogen; and p is 1 or 2 or 3; and Y is a group of formula IV wherein R^{d} is hydrogen or C₁₋₃ alkyl; and R^{e} is hydrogen or C₁₋₃ alkyl; and R^{f} is hydrogen or C₁₋₃ alkyl.

In a preferred embodiment of the invention B is a group of formula II wherein R^{b} and R^{c} can form together with the nitrogen, a 3-10 member heterocycloalkyl optionally substituted by C₁₋₆ alkyl; and D is a group of formula III wherein R^{a} is hydrogen; and p is 1; and Y is a group of formula IV wherein R^{d} is hydrogen; R^{e} is hydrogen; and R^{f} is C₁₋₃ alkyl.

In another preferred embodiment of the invention B is a group of formula II wherein wherein R^{b} is hydrogen; R^{c} is C₃₋₁₀ cycloalkyl; and D is a group of formula III wherein R^{a} is hydrogen; and p is 2; and Y is a group of formula V wherein R^{g} is C₁₋₃ alkyl.

In another preferred embodiment of the invention B is an aryl optionally substituted by C₁₋₆ alkyl; and D is a group of formula III wherein R^{a} is hydrogen; and p is 2; and Y is a group of formula V wherein R^{g} is C₁₋₃ alkyl.

In a more preferred embodiment of the invention B is aryl optionally substituted by halogen; and D is a group of formula III wherein R^{a} is hydrogen; and p is 1; and Y is a group of formula IV wherein R^{d} is hydrogen; and R^{e} is hydrogen; and R^{f} is hydrogen or C₁₋₃ alkyl.

In another more preferred embodiment of the invention B is C₃₋₁₀ cycloalkenyl optionally by C₁₋₆ alkyl group; and D is a group of formula III wherein R^{a} is hydrogen; and p is 1; and Y is a group of formula IV wherein R^{d} is hydrogen; and R^{e} is hydrogen; and R^{f} is C₁₋₃ alkyl.

Compounds of the invention are:
6-cyclohex-1-en-1-yl-4-[3-(methylamino)pyrrolidin-1-yl]pyridin-2-amine;
6-(4-chlorophenyl)-4-(4-methylpiperazin-1-yl)pyridin-2-amine;
6-(4-methylcyclohex-1-en-1-yl)-4-(4-methylpiperazin-1-yl)pyridin-2-amine;
6-adamantan-2-yl-4-(4-methylpiperazin-1-yl)pyridin-2-amine;
4-(3-aminopyrrolidin-1-yl)-6-cyclohex-1-en-1-ylpyridin-2-amine;
6-(3-methylphenyl)-4-(4-methylpiperazin-1-yl)pyridin-2-amine;
4-[(3R)-3-aminopyrrolidin-1-yl]-6-(4-chlorophenyl)pyridin-2-amine;
4-(4-methylpiperazin-1-yl)-6-(4-methylpiperidin-1-yl)pyridin-2-amine;
N-cycloheptyl-4-(4-methylpiperazin-1-yl)pyridine-2,6-diamine;
4-[3-(methylamino)pyrrolidin-1-yl]-6-(2-methylpyrrolidin-1-yl)pyridin-2-amine;
4-(4-methylpiperazin-1-yl)-6-(2-methylpyrrolidin-1-yl)pyridin-2-amine;
4-[3-(methylamino)pyrrolidin-1-yl]-6-(4-methylpiperidin-1-yl)pyridin-2-amine;
4-[(3-methylamino)pyrrolidin-1-yl]-6-(4-trifluoromethylphenyl)pyridin-2-amine;
4-[(3-methylamino)pyrrolidin-1-yl]-6-(4-trifluoromethoxyphenyl)pyridin-2-amine;
6-(4-chlorophenyl)-4-[(3-methylamino)pyrrolidin-1-yl)pyridin-2-amine;
4-[(3-methylamino)pyrrolidin-1-yl]-6-(3-methylphenyl)pyridin-2-amine.

Preferred compounds of the invention are:
N-cycloheptyl-4-(4-methylpiperazin-1-yl)pyridine-2,6-diamine;
4-[3-(methylamino)pyrrolidin-1-yl]-6-(2-methylpyrrolidin-1-yl)pyridin-2-amine;
4-[(3-methylamino)pyrrolidin-1-yl]-6-(3-methylphenyl)pyridin-2-amine.

More preferred compounds of the invention are:
6-cyclohex-1-en-1-yl-4-[3-(methylamino)pyrrolidin-1-yl]pyridin-2-amine;
6-(4-chlorophenyl)-4-[(3-methylamino)pyrrolidin-1-yl)pyridin-2-amine.

The “pharmaceutically acceptable salts” according to the invention include all therapeutically active, non-toxic acid salt forms which the compounds of formula (I) are able to form. The acid addition salt form of a compound of formula (I) that occurs in its free form as a base can be obtained by treating the free base with an appropriate acid such as an inorganic acid, for example, a hydrohalic such as hydrochloric, hydroiodic or hydrobromic, sulfuric, nitric, phosphoric and the like; or an organic acid, such as, for example, acetic, oxalic, p-bromophenylsulfonic, carbonic, benzoic, formic, propionic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, palmoic, and the like. Conversely said salt forms can be converted into the free forms by treatment with an appropriate base.

The “pharmaceutically acceptable salts” according to the invention include therapeutically active, non-toxic base salt forms which the compounds of formula I are able to form. For example, the compounds of formula I containing acidic protons may be converted into their therapeutically active, non-toxic base addition salt forms, e.g. metal or amine salts, by treatment with appropriate organic and inorganic bases. Appropriate base salt forms include, for example but are not limited to, ammonium salts, alkali and alkaline earth metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like. Conversely said, salt forms can be converted into the free forms by treatment with an appropriate acid.

Compounds of the formula I and their salts can be in the form of solvates, which are included within the scope of the present invention. Such solvates include for example hydrates, alcoholates and the like.

Some of the compounds of formula I and some of their intermediates have at least one stereogenic centre in their structure. This stereogenic centre may be present in a R or a S configuration, said R and S notation is used in correspondence with the rules described in Pure Appl. Chem., 45 (1976) 11-30.

The invention also relates to all stereoisomeric forms such as enantiomeric and diastereoisomeric forms of the compounds of formula I or mixtures thereof (including all possible mixtures of stereoisomers).

Some of the compounds of formula I may also exist in tautomeric forms. Such forms although not explicitly indicated in the above formula are intended to be included within the scope of the present invention.

With respect to the present invention reference to a compound or compounds is intended to encompass that compound in each of its possible isomeric forms and mixtures thereof unless the particular isomeric form is referred to specifically.

Compounds according to the present invention may exist in different polymorphic forms. Although not explicitly indicated in the above formula, such forms are intended to be included within the scope of the present invention.

The invention also includes within its scope prodrug forms of the compounds of formula I and its various sub-scopes and sub-groups.

The term “prodrug” as used herein includes compound forms, which are rapidly transformed in vivo to the parent compound according to the invention, for example, by hydrolysis in blood. Prodrugs are compounds bearing groups that are removed by biotransformation prior to exhibiting their pharmacological action. Such groups include moieties that are readily cleaved in vivo, from the compound bearing it, which compound after cleavage remains or becomes pharmacologically active. Metabolically cleavable groups form a class of groups well known to practitioners in the art. The compounds bearing the metabolically cleavable groups have the advantage that they may exhibit improved bioavailability as a result of enhanced solubility and/or rate of absorption conferred upon the parent compound by virtue of the presence of the metabolically cleavable group (T. Higuchi and V. Stella, “Pro-drugs as Novel Delivery System”, Vol. 14 of the A.C.S. Symposium Series; “Bioreversible Carriers in Drug Design”, ed. Edward B. Roche, American Pharmaceutical Association and Pergamon Press, 1987).

It has now been found that compounds of formula I and their pharmaceutically acceptable salts are useful in a variety of pharmaceutical indications. For example, the compounds according to the invention are useful for the treatment of inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin (pruritis); diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

Thus, the present invention, in a further aspect, concerns the use of a compound of formula I or a pharmaceutically acceptable salt thereof for the manufacture of a medicament for the treatment of disorders such as mentioned above.

In particular, the present invention concerns the use of a compound of formula I or a pharmaceutically acceptable salt thereof, for the manufacture of a medicament for the treatment of H₄ dependent such as inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin (pruritis) or diseases of the gastrointestinal tract including inflammatory bowel disease, Crohn's disease, ulcerative colitis or autoimmune diseases including rheumatoid arthritis, multiple sclerosis.

The compounds of the invention are useful for treating conditions in which there is an influx of leukocytes in the tissues. These conditions include inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin (pruritis) or diseases of the gastrointestinal tract including inflammatory bowel disease, Crohn's disease, ulcerative colitis or autoimmune diseases including rheumatoid arthritis, multiple sclerosis.

The compounds of the invention exhibit the biological activity by inhibiting the histamine binding to the H₄ receptor or on an activated H₄ receptor. Subjects in need of treatment for a H₄ dependent inflammatory disorder or inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin (pruritis) or diseases of the gastrointestinal tract including inflammatory bowel disease, Crohn's disease, ulcerative colitis or autoimmune diseases including rheumatoid arthritis, multiple sclerosis, can be treated by administering to the patient an effective amount of one or more of the above-identified compounds or a pharmaceutically acceptable derivative or salt thereof in a pharmaceutically acceptable carrier or diluent to reduce formation of oxygen radicals. The active materials can be administered by any appropriate route, for example, orally, parenterally, intravenously, intradermally, subcutaneously, intramuscularly or topically, in liquid, cream, gel or solid form, via a buccal or nasal spray, or aerosol.

The invention further concerns the use of the compounds of formula I for the manufacture of a medicament for therapeutic application.

In particular, the invention concerns the use of the compounds of formula I for the manufacture of a medicament useful for treating conditions in which there is likely to be a H₄ dependent inflammatory component.

The invention concerns the use of the compound of formula I for the manufacture of a medicament useful for treating inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal disfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

The invention further concerns the compounds of formula I for use as medicaments.

The invention concerns the compounds of formula I for use as a medicament for inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

The activity and properties of the active compounds, oral availability and stability in vitro or in vivo can vary significantly among the optical isomers of the disclosed compounds.

In a preferred embodiment, the active compound is administered in an enantiomerically enriched form, i.e., substantially in the form of one isomer. By the term “substantially” we understand greater or equal to 95% of the said isomer.

The present invention also concerns a method for treating H₄ dependent inflammatory conditions inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or diseases of the gastrointestinal tract such as inflammatory bowel disease, Crohn's disease, ulcerative colitis or autoimmune diseases such as rheumatoid arthritis, multiple sclerosis, atherosclerosis, skin diseases where there's an influx of inflammatory cells, cardiovascular diseases, in a mammal in need of such treatment, comprising administering a therapeutic dose of at least one compound of formula I or a pharmaceutically acceptable salt thereof to a patient.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 0.01 to 1000 mg, preferably 0.05 to 500 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The activity of the compounds of formula I or their pharmaceutically acceptable salts, as H₄ antagonists can be determined in a tritiated histamine binding assay and in a H₄ GTP [symbol] S³⁵ binding assay. The objective of this test is to evaluate the anti-H₄ potential of a compound by measuring its inhibitory effect on histamine binding to the H₄ receptor or on H₄ receptor activation. Results obtained with compounds of formula I are indicative of a strong pharmacological effect.

For treating diseases, compounds of formula I or their pharmaceutically acceptable salts, may be employed at an effective daily dosage and administered in the form of a pharmaceutical composition.

Therefore, another embodiment of the present invention concerns a pharmaceutical composition comprising an effective amount of a compound of formula I or a pharmaceutically acceptable salt thereof in combination with a pharmaceutically acceptable diluent or carrier.

To prepare a pharmaceutical composition according to the invention, one or more of the compounds of formula I or a pharmaceutically acceptable salt thereof, is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, or parenteral.

Pharmaceutical compositions comprising compounds according to the invention can, for example, be administered orally or parenterally, i.e., intravenously, intramuscularly, subcutaneously, transdermally, intrathecally or by inhalation.

Pharmaceutical compositions suitable for oral administration can be solids or liquids and can, for example, be in the form of tablets, pills, dragees, gelatine capsules, solutions, syrups, suppositories, patches, inhalants, and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions, which can release the active substance in a controlled manner. Pharmaceutical compositions, which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula I in compositions for oral administration is at least 0.5% by weight and can be up to 80% by weight with respect to the total weight of the composition.

For the preferred oral compositions, the daily dosage is in the range 0.01 to 1000 milligrams (mg) of compounds of formula I. In compositions for parenteral administration, the quantity of compound of formula I present is at least 0.5% by weight and can be up to 33% by weight with respect to the total weight of the composition. For the preferred parenteral compositions, the dosage unit is in the range 0.01 mg to 1000 mg of compounds of formula I.

The daily dose can fall within a wide range of dosage units of compound of formula I is generally in the range 0.01 to 1000 mg. However, it should be understood that the specific doses could be adapted to particular cases depending on the individual requirements, at the physician's discretion.

The compounds of the invention may be co-administered with another therapeutic agent most likely from a different therapeutic area.

Co-administration in this context means the dosing either of components, which are formulated together as a single dosage form; or the administration of separately formulated agents at substantially the same time, or sequential dosing of a compound of the invention followed by a therapeutic agent of a different therapeutic area.

In this context suitable examples of therapeutic agents may include, but are not limited to, histamine H1 antagonists such as cetirizine, histamine H2 antagonists, histamine H3 antagonists, leukotriene antagonists, PDE4 inhibitors such as 3-cyclopropylmethoxy-4-difluoromethoxy-N-[3,5-di-chloropyrid-4-yl]benzamide, muscarinic M3 antagonists, [symbol] ₂ agonists, theophylline, sodium chromoglycate, anti-TNF antibodies such as certolizumab pegol or adalimumab, anti-IL6 antibodies, anti-IL17 antibodies, adhesion molecule inhibitors, inhibitors of cytokine synthesis such as P38 MAP kinase inhibitors and inhibitors of PI3 kinase, methotrexate.

The present invention concerns also processes for preparing the compounds of formula I.

The compounds of formula I according to the invention can be prepared analogously to conventional methods as understood by the person skilled in the art of synthetic organic chemistry.

The following processes description sets forth certain synthesis routes in an illustrative manner. Other alternative and/or analogous methods will be readily apparent to those skilled in this art.

Compounds of formula I may be prepared according to one of the following general methods.

The synthesis of the compounds of the invention can be done by starting from a 2,4,6-trifluoro or 2,4,6-trichloropyridine. The coupling of the D moiety, may be achieved by reaction of 2,4,6-trifluoro or 2,4,6-trichloropyridine with H-D for example, 1-methylpiperazine, in the presence of a base such as triethylamine in a solvent such as NMP at a temperature from 0° C. to 200° C. “X” represents either chloro or fluoro.

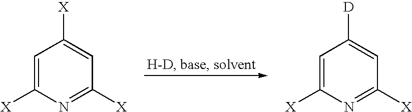

Introduction of the B group to the dichloro or difluoropyridine can be achieved by reaction with B-M where

M represents a metal such as zinc or magnesium, for example 2-adamantyl zinc bromide, or a boronic acid, for example 4-chlorophenylboronic acid, in the presence of a catalyst such as a palladium catalyst.

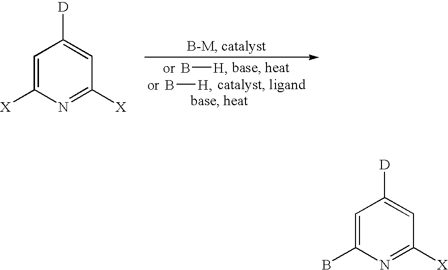

Subsequent introduction of the amino function can then be achieved by displacement of the remaining halogen with ammonia or a protected equivalent, for example 4-methoxybenzylamine, under direct thermal conditions, or under palladium catalysis.

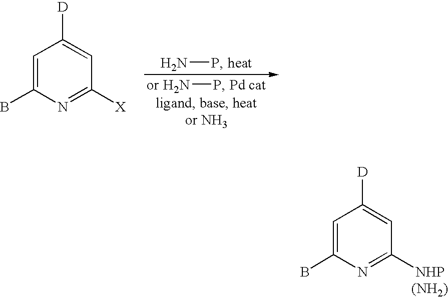

The synthesis of the compounds of the invention can also be done by starting from 2,6-dichloro-4-nitropyridine or 2,6-dibromo-4-nitropyridine. D can be introduced by reaction of H-D, for example 3-(N-methyl-N-tertbutoxycarbonylamino)pyrrolidine, in the presence of a base such as triethylamine in a solvent such as NMP at a temperature from 0° C. to 200° C. In this case “X” represents either chloro or bromo.

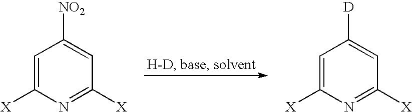

The introduction of a protected amino group can be done at position 2, by displacing the halogen, using an amine (H₂N—P where P is a protecting group), for example 4-methoxybenzylamine, heating under conventional or microwave conditions, or in the presence of a catalyst such as a palladium catalyst and a ligand such as a hindered phosphine in the presence of a base such as sodium tert-butoxide. Suitable amines include ammonia and 4-methoxybenzylamine. By protecting group we refer to a functional group that masks the characteristic reactivity of another group to which it can later be converted. Examples of H₂N—P include 4-methoxybenzylamine, 2,4-dimethoxybenzylamine, allylamine, di-allylamine, bis-trimethylsilylamine and the like.

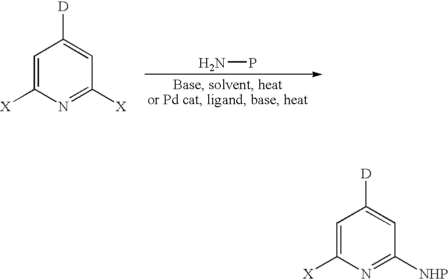

When the B group is aryl, C₃₋₁₀ cycloalkyl, or C₃₋₁₀ cycloalkenyl, its introduction can be achieved by reaction of the chloro- or fluoro- or bromopyridine (“X” is chloro or bromo or fluoro) with a reagent B-M, where M represents a metal such as zinc or magnesium, for example 2-adamantyl zinc bromide or a boronic acid, for example 4-chlorophenylboronic acid, in the presence of a catalyst such as a palladium catalyst.

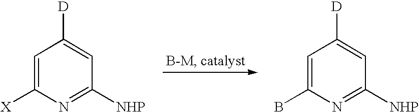

When the B group is according to formula II, its introduction is achieved by heating the halopyridine with a reagent B—H, for example cycloheptylamine, in the presence of a base under microwave or conventional conditions, or in the presence of a catalyst, for example a palladium reagent and a ligand such as a hindered phosphine.

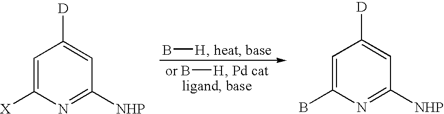

Removal of protecting groups, where appropriate, is achieved by treatment of the protected molecules with suitable reagents such as an acid, for example trifluoroacetic acid at a temperature from 0° C. to reflux, or for example by hydrogenation of benzylic protecting groups. For more details concerning deprotection methods, see “Protective Groups in Organic Chemistry”, Chapter 2, J. F. W. Omie, Plenum Press, London and New York, 1973 and “Protective Groups in Organic Synthesis”, Chapter 7, Th. W. Greene, John Wiley & Sons, 1999.

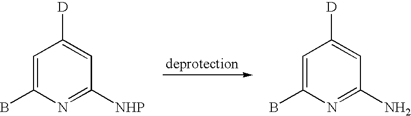

An alternative approach to the building of compounds of the invention is via introduction of the D group, such as 1-methylpiperazine to a di-halopyridine, for example 2-chloro-4-bromopyridine. The resulting di-substituted pyridine can then be metallated using an alkyl lithium reagent such as n-butyllithium in the presence of a coordinating reagent such as N,N-dimethylethanolamine and reacted with a reagent B—Y, where Y represents a leaving group such as a halogen, for example bromocyclohexane. The halopyridine is then reacted with ammonia or a protected equivalent and deprotected to give the final compounds.

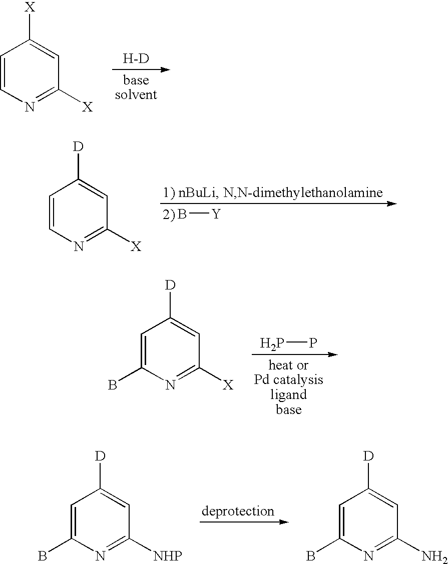

Similarly, a monoholopyridine containing the D group at position 4 may be deprotonated with n-butyllithium (n-BuLi) in the presence of a coordinating reagent such as N,N-dimethylethanolamine (Et₂NCH₂CH₂OH) and the resulting anion reacted with a halogenating agent such as hexachloroethylene (Cl₃C)₂ to give a di-halo pyridine. This may then be reacted to introduce an optionally protected amino group at position 2, the remaining halogen then reacted with a reagent B-M or B—H to introduce the B group at position 6. Subsequent deprotection, where required, then affords compounds of the invention.

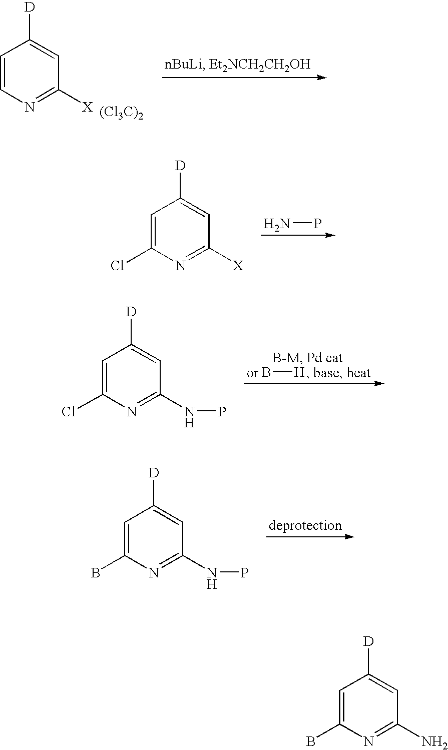

The present invention also relates to synthetic intermediates geometrical isomers, enantiomers, diastereoisomers, pharmaceutically acceptable salts and all possible mixtures thereof.

Specific synthetic intermediates are selected from the group consisting of:
2,6-difluoro-4-(4-methylpiperazinyl)pyridine;
2-fluoro-4-(4-methylpiperazinyl)-6-(4-methoxybenzylamino)pyridine;
2,6-difluoro-4-((3-N-methyl-N-tertbutoxycarbonylamino)pyrrolidino)-pyridine;
2-(4-methoxybenzylamino)-4-((3-N-methyl-N-tertbutoxycarbonylamino)pyrrolidino)-6-fluoropyridine;
2,6-dibromo-4-((3-tertbutoxycarbonylamino)pyrrolidino)-pyridine;
2-bromo-4-((3-N-methyl,N-tertbutoxycarbonylamino)pyrrolidino)-6-(4-methoxybenzylamino)-pyridine;
2,6-dichloro-4-((N-tertbutoxycarbonylamino)pyrrolidino)pyridine;
2-chloro-6-(4-methoxybenzylamino)-4-((N-tertbutoxycarbonylamino)pyrrolidino)pyridine;
2-chloro-4-(4-methylpiperazin-1-yl)-6-(4-methoxybenzylamino)pyridine;
2-(2-adamantyl)-4-(4-methylpiperazin-1-yl)-6-(4-methoxybenzylamino)pyridine.

The following examples are provided for illustrative purposes only and are not intended, nor should they be construed, as limiting the invention in any manner. Those skilled in the art will appreciate that routine variations and modifications of the following examples can be made without exceeding the spirit or scope of the invention.

Unless specified otherwise in the examples, characterization of the compounds is performed according to (LCMS) liquid chromatography mass spectra, preparative liquid chromatography LC, NMR, and silica gel chromatography methods.

NMR spectra are recorded on Bruker AV300 and DRX 400 spectrometers at 300 and 400 MHz respectively.

Chromatographic separations are performed on Davis 5 [symbol] M silica gel.

The Waters mass spectrometers used are of model ZMD or ZQ both Waters.

Various reactions took place in an Emrys Optimiser microwave reactor.

The following abbreviations are used in the examples:

### DCM—Dichloromethane

### DIPEA—N, N-Diisopropylethylamine

DMSO—Dimethyl sulphoxide

### DMF—N,N-Dimethylformamide

d₆-DMSO—Dimethyl-d₆ sulphoxide

### DPPF—1,1′-Bis(diphenylphosphino)ferrocene

### MeOH Methanol

d₄-MeOH—Methanol-d₄
NMP—1-Methyl-2-pyrrolidinone
NH₄OH—ammonium hydroxide
MTBE—Methyl tert-butyl ether
RT—Retention time

### TEA or Et₃N—Triethylamine;

TFA—Trifluoroacetic acid
EtOAc—Ethyl acetate
EtOH—ethanol

### THF—Tetrahydrofuran

ESI—Electrospray ionisation

### Pos—Positive

### Neg—Negative

The IUPAC names of compounds were generated using ACD (Labs Release: 9.00, product version: 9.04).

All the reagents, solvents, catalysts for which the synthesis is not described have been purchased from chemical vendors such Sigma Aldrich, Fluka, Lancaster, however some known reaction intermediates, for which the registry numbers (RN) are mentioned, have been prepared in-house following known procedures.

The LCMS conditions used to obtain the retention times (RT) are described herein:

### 1. LCMS Conditions

HP1100 (Diode Array) linked to a Finnigan LC-Q Mass Spectrometer, ESI mode with Pos/Neg ionisation or Waters 2695 linked to a Waters ZMD Mass Spectrometer, ESI mode with Pos/Neg ionisation.

| Column: | Phenomenex Luna C18(2) 100 × 4.6 mm, | |
|---|---|---|
| | 5 μm particle size Analytical column | |
| Column temp: | 35° C. | |
| Mobile phase: | A: Water + 0.08% formic acid | |
| | B: Acetonitrile + 0.08% formic acid | |
| Flow rate: | 3 ml/min | |
| | Time (min) | % Composition B |
| Gradient: | 0 | 5 |
| | 4.40 | 95 |
| | 5.30 | 95 |
| | 5.32 | 5 |
| | 6.50 | 5 |

| | |
|---|---|
| Run time: | 6.50 min |
| Typical injection volume: | 10 μl |
| Detector wavelength: | DAD 200-400 nm |

### 2. LCMS Conditions (pH 5.8)

HP1100 (Diode Array) linked to a Finnigan LC-Q Mass Spectrometer, ESI mode with Pos/Neg ionisation or Waters 2695 linked to a Waters ZMD Mass Spectrometer, ESI mode with Pos/Neg ionisation.

| Column: | Phenomenex Luna C18(2) 100 × 4.6 mm, | |
|---|---|---|
| | 5 μm particle size Analytical column | |
| Column temp: | 35° C. | |
| Mobile phase: | A: 5 mM NH₄OAc pH 5.8 | |
| | B: 95:5, MeCN: 100 mM NH₄OAc pH 5.8 | |
| Flow rate: | 3 ml/min | |
| | Time (min) | % Composition B |
| Gradient: | 0 | 5 |
| | 4.40 | 95 |
| | 5.30 | 95 |
| | 5.32 | 5 |
| | 6.50 | 5 |

| | |
|---|---|
| Run time: | 6.50 min |
| Typical injection volume: | 10 μl |
| Detector wavelength: | DAD 200-400 nm |

The following preparative LC conditions were used to purify compounds as described herein:

### Preparative LC Conditions (pH 2.5) (Method A)

Waters autopreparative mass and UV directed: ZQ mass spectrometer, 996 PDA, 2525 pump and 2767 autosampler/fraction collector and 2757 fraction collector.

| | |
|---|---|
| Column: | Phenomenex Luna C18(2) 250 × 21.2 mm, |
| | 5 μm particle size prep column |
| Column temp: | Ambient |
| Mobile phase: | A: Water + 0.08% formic acid |
| | B: Acetonitrile + 0.08% formic acid |
| Flow rate: | 25 ml/min |
| Gradient: | Variable - depends on retention time |
| | of sample in LC-MS analysis |
| Run time: | 20 min |
| Injection volume: | 1 ml at 50 mg/ml (typically) |
| Detector wavelength: | 200 to 400 nm |

### Preparative LC Conditions (pH 5.8) (Method B)

Waters autopreparative mass and UV directed: ZQ mass spectrometer, 996 PDA, 2525 pump and 2767 autosampler/fraction collector and 2757 fraction collector.

| | |
|---|---|
| Column: | Phenomenex Luna C18(2) 250 × 21.2 mm, |
| | 5 μm particle size prep column |
| Column temp: | Ambient |
| Mobile phase: | A: 10 mM ammonium acetate pH 5.8 |
| | B: 5:95, 200 mM ammonium acetate |
| | pH 5.8: Acetonitrile |
| Flow rate: | 25 ml/min |
| Gradient: | Variable - depends on retention time |
| | of sample in LC-MS analysis |
| Run time: | 20 min |
| Injection volume: | 1 ml at 50 mg/ml (typically) |
| Detector wavelength: | 200 to 400 nm |

### SYNTHETIC EXAMPLES

### Example 1

### Synthesis of 2,6-difluoro-4-(4-methylpiperazinyl)pyridine (Intermediate 1)

A solution of 2,4,6-trifluoropyridine [CAS 3512-17-2] (3.7 g), 1-methylpiperazine [CAS 109-01-3] (3.5 g) in EtOH (30 ml) and Et₃N (4.5 ml) is stirred at room temperature for 1 h. The mixture is added to water (100 ml) and the product collected by filtration to give the title compound as colorless solid (3.10 g, 52%). LCMS 214 [M+H]⁺, RT (pH 5.8) 2.34 mins. ¹H NMR 300 MHz (CDCl₃) (δ ppm): 6.10 (2H, s), 3.35 (4H, m), 2.55 (4H, m), 2.32 (3H, s).

### Example 2

### Synthesis of 2-fluoro-4-(4-methylpiperazinyl)-6-(4-methoxybenzylamino)pyridine (Intermediate 2)

A solution of Intermediate 1 (300 mg) and 4-methoxybenzylamine [CAS 2393-23-9] (500 mg) in NMP (3 ml) and Et₃N (500 mg) is heated under microwave conditions at 200° C. for 1 h, the mixture is added to water (30 ml) and extracted with EtOAc. The solvent is washed with water (2×20 ml), dried and evaporated and the residue purified by column chromatography eluting with 2% MeOH/DCM/0.5% NH₄OH to give the title compound as beige solid (280 mg). LCMS 331 [M+H]⁺, RT (pH 5.8) 2.85 mins. ¹H NMR 300 MHz (CDCl₃) (δ ppm): 7.28 (2H, d), 6.85 (2H, d), 5.65 (1H, m), 5.50 (1H, m), 4.68 (1H, t), 4.36 (2H, d), 3.70 (3H, s), 3.25 (4H, m), 2.50 (4H, m), 2.33 (3H, s).

### Example 3

### Synthesis of 2,6-difluoro-4-((3-N-methyl-N-tertbutoxycarbonylamino)pyrrolidino)-pyridine (Intermediate 3)

A solution of 2,4,6-trifluoropyridine [CAS 3512-17-2] (1.1 g) is stirred at room temperature with 3-(N-methyl-N-Boc amino)pyrrolidine [CAS 169750-01-0] (1.6 g) in ethanol (10 ml) and triethylamine (1.5 ml) for 2 h, then the mixture is added to water (30 ml) and extracted with EtOAc (2×20 ml). The organic solvent is washed with water (30 ml), dried and evaporated. The crude product is purified by chromatography (3:1 heptane/EtOAc) to give the title compound as colourless solid (1.50 g). LCMS 314 [M+H]⁺, RT (pH 2) 3.92 mins. ¹H NMR 300 MHz (CDCl₃) (δ ppm): 5.80 (2H, s), 4.87 (1H, br s), 3.50 (2H, m), 3.35 (1H, m), 3.20 (1H, m), 2.80 (3H, s), 2.10-2.30 (2H, m), 1.47 (9H, s).

### Example 4

### Synthesis of 2-(4-methoxybenzylamino)-4-((3-N-methyl-N-tertbutoxycarbonylamino)pyrrolidino)-6-fluoropyridine (Intermediate 4)

A solution of intermediate 3 (110 mg) is heated under microwave irradiation with 4-methoxybenzylamine [CAS 2393-23-9] (62 mg) and triethylamine (100 mg) in NMP (1 ml) at 170° C. for 2 h, then the solution is added to water (10 ml) and extracted with EtOAc (10 ml). The organic solvent is washed with water (10 ml), dried and evaporated and the crude product purified by chromatography (2:1 heptane/EtOAc) to give the title compound as beige solid (40 mg). LCMS 431 [M+H]⁺, RT (pH 2) 4.14 mins. ¹H NMR 300 MHz (CDCl₃) (δ ppm): 7.28 (2H, d), 7.85 (2H, d), 5.40 (1H, s), 5.25 (1H, s), 4.86 (1H, br s), 4.63 (1H, t), 4.33 (2H, d), 3.71 (3H, s), 3.40 (2H, m), 3.25 (1H, m), 3.16 (1H, m), 2.75 (3H, s), 2.00-2.20 (2H, m), 1.45 (9H, s).

### Example 5

### Synthesis of 2,6-dibromo-4-((3-N-methyl-N-tertbutoxycarbonylamino)-pyrrolidino)-pyridine (Intermediate 5)

A solution of 2,6-dibromo-4-nitropyridine [CAS 175422-04-5] (0.28 g) and 3-(N-methyl-N-tertbutoxycarbonylamino)pyrrolidine [CAS 172478-00-1] (0.20 g) in a mixture of NMP (3 ml) and triethylamine (0.2 ml) is heated under microwave irradiation at 100° C. for 1 h, then cooled and added to water (20 ml). The mixture is extracted with EtOAc (20 ml), the organic solvent washed with water (2×10 ml), dried and evaporated. The residue is purified by column chromatography (1:1 ether/heptane) to give the title compound as colorless solid (0.33 g). LCMS 435 [M+H]⁺, RT (pH 2.5) 4.31 mins. ¹H NMR 300 MHz (CDCl₃) (δ ppm): 6.52 (2H, s), 4.87 (1H, br s), 3.48 (2H, m), 3.30 (1H, m), 3.20 (1H, m), 2.80 (3H, s), 2.10-2.30 (2H, m), 1.49 (9H, s).

Compounds Int. 11 and Int. 13 are prepared according to the method described in Example 5.
The reagents used and the results obtained are tabulated below.

| | | | M⁺ | ¹H NMR |
|---|---|---|---|---|
| | | | (M⁺ + 1) | (solvent, |
| Int. No | IUPAC Name | Starting materials | Observed | δ ppm) |
| Int. 11 | tert-Butyl 4-(2,6- | 2,6-Dibromo-4- | 422 [M + H⁺] | (CDCl₃) 6.73 (2H, s), |
| | dibromopyridin-4- | nitropyridine [CAS | RT 4.14 min | 3.57 (4H, m), 3.32 (4H, m), |
| | yl)piperazine-1- | 175422-04-5], tert- | (pH 2) | 1.48 (9H, s) |
| | carboxylate | butyl piperazine-1- | | |
| | | carboxylate [CAS | | |
| | | 57260-71-6] | | |
| Int. 13 | tert-Butyl [1-(2,6- | 2,6-Dibromo-4- | 408 [M + H]⁺ | (CDCl₃) 6.34 (2H, s), |
| | dibromopyridin-4- | nitropyridine [CAS | RT 3.92 mins | 5.00 (1H, br s), 4.65 (1H, m), |
| | yl)azetidin-3- | 175422-04-5], tert- | (pH 2) | 4.25 (2H, dd), 3.78 (2H, dd), |
| | yl]carbamate | butyl azetidin-3- | | 1.44 (9H, s) |
| | | ylcarbamate [CAS | | |
| | | 91188-13-5] | | |

*Int. No means Intermediate Number

### Example 6

### Synthesis of 2-bromo-4-((3-N-methyl,N-tertbutoxycarbonylamino)pyrrolidino)-6-(4-methoxybenzylamino)pyridine (Intermediate 6)

A solution of Intermediate 5 (800 mg) and 4-methoxybenzylamine [CAS 2393-23-9] (2.6 g) in NMP (5 ml) is heated under microwave irradiation at 160° C. for 2 h. The mixture is added to water (30 ml) and extracted with EtOAc (2×20 ml). The combined organic solvent is washed with water (2×20 ml), dried and evaporated and the residue purified by chromatography (3:2 ether/heptane) to give the title compound as beige solid (410 mg). LCMS 491 [M+H]⁺, RT (pH 2.5) 3.52 mins. ¹H NMR 300 MHz (CDCl₃) (δ ppm): 7.25 (2H, d), 6.85 (2H, d), 6.03 (1H, s), 5.28 (1H, s), 4.80 (1H, m), 1.78 (1H, t), 4.30 (2H, d), 3.80 (3H, s), 3.40 (2H, m), 3.25 (1H, m), 3.15 (1H, m), 2.78 (3H, s), 2.00-2.20 (2H, m), 1.47 (9H, s).

Compounds Int. 12 and Int. 14 are prepared according to the method described in Example 6.
The reagents used and the results obtained are tabulated below.

| | | | M⁺ | ¹H NMR |
|---|---|---|---|---|
| | | | (M⁺ + 1) | (solvent, |
| Int. No | IUPAC Name | Starting materials | Observed | δ ppm) |
| Int 12 | tert-Butyl 4-{2- | Int 11, and | 477/ | (CDCl₃) 7.25 (2H, d), |
| | bromo-6-[(4- | 4-methoxybenzyl- | 479 [M + H]⁺ | 6.86 (2H, d), 6.28 (1H, m), |
| | methoxybenzyl)amino]pyridin- | amine | RT 4.96 mins | 5.52 (1H, m), 4.82 (1H, br t), |
| | 4- | [CAS 2393-23-9] | (pH 2) | 4.33 (2H, d), 3.80 (3H, s), |
| | yl}piperazine-1- | | | 3.50 (4H, m), 3.21 (4H, m), |
| | carboxylate | | | 1.46 (9H, s) |
| Int. 14 | tert-Butyl (1-{2- | Int 13, and | 463/ | (CDCl₃) 7.25 (2H, d), |
| | bromo-6-[(4- | 4-methoxybenzyl- | 465 [M + H]⁺ | 6.86 (2H, d), 5.88 (1H, m), |
| | methoxybenzyl)amino]pyridin- | amine | RT 3.29 min | 5.12 (1H, m), 4.91 (1H, br s), |
| | 4- | [CAS 2393-23-9] | (pH 2) | 4.82 (1H, br t), 4.55 (1H, m), |
| | yl}azetidin-3- | | | 4.29 (2H, d), 4.16 (2H, dd), |
| | yl)carbamate | | | 3.80 (3H, s), 3.63 (2H, dd), |
| | | | | 1.44 (9H, s) |

### Example 7

### Synthesis of 2,6-dichloro-4-((N-tertbutoxycarbonylamino)pyrrolidino)pyridine (Intermediate 7)

A solution of 2,4,6-trichloropyridine (4 g) and 3-tertbutoxycarbonylaminopyrrolidine [CAS 99724-19-3] (4 g) is heated under microwave irradiation in a mixture of NMP (5 ml) and Et₃N (5 ml) at 100° C. for 1 h, then the mixture is added to water (100 ml) and extracted with EtOAc (50 ml). The solvent is washed with water, dried and evaporated and the crude product purified by chromatography (4:1 heptane/EtOAc) to give the title compound as colourless solid (1.8 g). LCMS 333 [M+H]⁺, RT (pH 2.5) 3.78 mins. ¹H NMR 300 MHz (CDCl₃) (δ ppm): 6.32 (2H, s), 4.70 (1H, br s), 4.35 (1H, m), 3.63 (1H, m), 3.40 (2H, m), 3.18 (1H, m), 2.23 (1H, m), 2.00 (1H, m), 1.45 (9H, s).

### Example 8

### Synthesis of tert-butyl [(3R)-1-(2,6-dichloropyridin-4-yl)pyrrolidin-3-yl]-carbamate (Intermediate 15)

Intermediate 15 is prepared from 2,4,6-trichloropyridine [CAS 16063-69-7] (250 mg) and tent-butyl (3R)-pyrrolidin-3-ylcarbamate [CAS 122536-77-0] (200 mg) in a similar manner to the method described for Intermediate 7, to give the title compound as colourless solid (300 mg, 84%). LCMS 332 [M+H]⁺, RT 3.94 mins (pH 5.8). ¹H NMR 300 MHz (CDCl₃) (δ ppm): 6.32 (2H, s), 4.70 (1H, br s), 4.35 (1H, m), 3.63 (1H, m), 3.40 (2H, m), 3.18 (1H, m), 2.23 (1H, m), 2.00 (1H, m), 1.45 (9H, s).

### Example 9

### Synthesis of 2-chloro-6-(4-methoxybenzylamino)-4-((N-tertbutoxycarbonyl amino)pyrrolidino)pyridine (Intermediate 8)

A mixture of Intermediate 7 (1.20 g), 4-methoxybenzylamine [CAS 2393-23-9] (1.0 g) and cesium carbonate [CAS 534-17-8] (2.5 g) in toluene (10 ml) is de-oxygenated by passage of nitrogen gas for 30 min, then palladium acetate [CAS 3375-31-3] (10 mg) and dppp (15 mg) is added and the mixture heated under microwave irradiation at 140° C. for 4 h. The solution is added to water (30 ml) and extracted with EtOAc (2×20 ml). The organic solvent is dried and evaporated and the residue purified by chromatography (2:1 heptane/EtOAc) to give the title compound as beige solid (180 mg). LCMS 433 [M+H]⁺, RT (pH 2.5) 3.10 mins. ¹H NMR 300 MHz (CDCl₃) (δ ppm): 7.28 (2H, d), 6.87 (2H, d), 5.85 (1H, s), 5.25 (1H, s), 4.80 (1H, t), 4.67 (1H, br s), 4.32 (2H, d), 4.28 (1H, m), 3.80 (3H, s), 3.55 (1H, m), 3.30 (2H, m), 3.10 (1H, m), 2.23 (1H, m), 1.90 (1H, m), 1.45 (9H, s).

### Example 10

### Synthesis of tert-butyl [(3R)-1-{2-chloro-6-[(4-methoxybenzyl)amino]pyridin-4-yl}pyrrolidin-3-yl]-carbamate (Intermediate 16)

Intermediate 16 is prepared from Intermediate 15 (150 mg) and 4-methoxybenzylamine [CAS 2393-23-9], in a similar manner to the method described for Intermediate 8, to give the title compound as beige solid (35 mg, 18%). LCMS 433/435 [M+H]⁺, RT 3.10 mins (pH 2.5). ¹H NMR 300 MHz (CDCl₃) (δ ppm): 7.28 (2H, d), 6.87 (2H, d), 5.85 (1H, s), 5.25 (1H, s), 4.80 (1H, t), 4.67 (1H, br s), 4.32 (2H, d), 4.28 (1H, m), 3.80 (3H, s), 3.55 (1H, m), 3.30 (2H, m), 3.10 (1H, m), 2.23 (1H, m), 1.90 (1H, m), 1.45 (9H, s).

### Example 11

### Synthesis of 2-chloro-4-(4-methylpiperazin-1-yl)-6-(4-methoxybenzylamino)pyridine (Intermediate 9)

A solution of 2,6-dichloro-4-(4-methylpiperazin-1-yl)pyridine [CAS 881418-26-4] (200 mg) is heated under microwave irradiation with 4-methoxybenzylamine [CAS 2393-23-9] (200 mg) in a mixture of NMP (1 ml) and Et₃N (200 mg) at 250° C. for 30 min. The solution is added to water (10 ml) and extracted with EtOAc (2×10 ml). The organic solvent is washed with water (2×10 ml), dried and evaporated and the residue purified by prep HPLC (method B) to give the title compound as beige solid (60 mg). LCMS 347 [M+H]⁺, RT (pH 5.8) 3.12 mins. ¹H NMR 300 MHz (CDCl₃) (δ ppm): 7.30 (2H, d), 6.90 (2H, d), 6.18 (1H, s), 5.58 (1H, s), 4.82 (1H, t), 4.36 (2H, d), 3.83 (3H, s), 3.29 (4H, m), 2.50 (4H, m), 2.35 (3H, s).

### Example 12

### Synthesis of 2-(2-adamantyl)-4-(4-methylpiperazin-1-yl)-6-(4-methoxybenzylamino)pyridine (Intermediate 10)

A solution of Intermediate 9 (40 mg) is heated at reflux with 2-adamantyl zinc bromide [CAS 171860-65-4] (0.5 M in THF, 1 ml) and Pd(dppf)Cl₂ [CAS 72287-26-4] in THF (10 ml) for 18 h. The mixture is evaporated and the residue purified by chromatography (3% MeOH/DCM 1% NH₄OH) to give the title compound as beige solid (25 mg). LCMS 447 [M+H]⁺, RT (pH 5.8) 3.47 mins. ¹H NMR 300 MHz (CDCl₃) (δ ppm): 7.30 (2H, d), 6.87 (2H, d), 6.22 (1H, s), 5.60 (1H, s), 4.75 (1H, br s), 4.40 (2H, d), 3.83 (3H, s), 3.29 (4H, m), 2.88 (1H, m), 2.55 (2H, m), 2.53 (4H, m), 2.33 (3H, s), 1.50-2.00 (12H, m).

### Example 13

### Synthesis of 2-amino-4-(4-methylpiperazinyl)-6-(4-methylpiperidinyl)pyridine (Compound 1)

A solution of Intermediate 2 (180 mg) and 4-methylpiperidine [CAS 626-58-4] (1 ml) is heated under microwave irradiation at 220° C. for 2 h. The mixture is added to water (10 ml) and extracted with EtOAc. The solution is washed with water (2×10 ml), dried and evaporated to a colorless oil which is then dissolved in TFA (10 ml) and heated at reflux for 1 h. The solution is evaporated in vacuo and purified by preparative HPLC (Method B) to give the title compound as beige solid (50 mg). LCMS 290 [M+H]⁺, RT (pH 5.8) 2.16 mins. ¹H NMR 300 MHz (CDCl₃) (δ ppm): 5.55 (1H, m), 5.40 (1H, m), 2.10-4.20 (2H, m), 4.05 (2H, br s), 3.25 (4H, m), 2.60-2.75 (2H, m), 2.50 (4H, m), 2.32 (3H, s), 1.40-1.60 (3H, m), 1.15-1.28 (2H, m), 0.96 (3H, d).

Compounds 2 and 3 are prepared according to the method described in Example 13.
The reagents used and the results obtained are tabulated below.

| | | | M⁺ | ¹H NMR |
|---|---|---|---|---|
| Comp. | | | (M⁺ + 1) | (solvent, |
| No | IUPAC Name | Starting materials | Observed | δ ppm) |
| 2 | N-cycloheptyl-4- | Intermediate 2 | 304 [M + H⁺] | CDCl₃ 5.35 (1H, m), 5.20 (1H, |
| | (4- | and | RT 2.28 mins | m), 4.25 (1H, d), 4.05 (2H, br |
| | methylpiperazin-1- | cycloheptylamine | (pH | s), 3.50 (1H, m), 3.25 (4H, m), |
| | yl)pyridine-2,6- | [CAS 5452-35-7] | 5.8) | 2.50 (4H, m), 2.32 (3H, s), |
| | diamine | | | 1.90-2.15 (4H, m), |
| | | | | 1.30-1.75 (8H, m) |
| 3 | 4-(4- | Intermediate 2 | 276 [M + H⁺] | CDCl₃ 5.35 (1H, m), 5.22 (1H, |
| | methylpiperazin-1- | and | RT 1.93 min | m), 4.18 (2H, br s), 4.10 (1H, |
| | yl)-6-(2- | 2-methyl- | (pH 5.8) | m), 3.50 (1H, m), 3.30 (1H, m), |
| | methylpyrrolidin-1- | pyrrolidine | | 3.22 (4H, m), 2.50 (4H, m), |
| | yl)pyridin-2-amine | [CAS 765-38-8] | | 2.32 (3H, s), 1.85-2.10 (3H, m), |
| | | | | 1.65 (1H, m), 1.18 (3H, d) |

*Comp. No means Compound Number

### Example 14

### Synthesis of 2-amino-4-((3-N-methylamino)pyrrolidino)-6-(4-methylpiperidinyl)pyridine (Compound 4)

A solution of intermediate 4 (30 mg) is heated in 4-methylpiperidine [CAS 626-28-4] (1 ml) under microwave irradiation at 150° C. for 1 h. The mixture is filtered through silica eluting with 2% MeOH/DCM and the crude product dissolved in TFA (2 ml) and heated at reflux for 2 h. The solvent is evaporated and the residue purified by prep HPLC (Method B) to give the title compound as colorless solid (1.5 mg). LCMS 290 [M+H]⁺, RT (pH 5.8) 1.94 mins. ¹H NMR 300 MHz (d₄-MeOH) (δ ppm): 5.00 (1H, s), 4.97 (1H, s), 3.95 (2H, m), 3.55 (1H, m), 3.35 (1H, m), 3.15 (1H, m), 2.73 (2H, m), 2.45 (3H, s), 2.20 (1H, m), 1.95 (1H, m), 1.70 (2H, m), 1.60 (1H, m), 1.25 (2H, m), 1.00 (3H, d)

Compound 5 is prepared according to the method described in Example 14.

The reagents used and the results obtained are tabulated below.

| | | | M⁺ | ¹H NMR |
|---|---|---|---|---|
| Comp. | | | (M⁺ + 1) | (solvent, |
| No | IUPAC Name | Starting materials | Observed | δ ppm) |
| 5 | 4-[3- | Intermediate 4 | 276 [M + H⁺] | CDCl₃ 5.12 (1H, s), 4.95 (1H, |
| | (methylamino)pyrrolidin- | and | RT 1.51 min | s), 4.10 (1H, m), 3.96 (2H, br |
| | 1-yl]-6-(2- | 2-methyl- | (pH 5.8) | s), 3.45 (2H, m), 3.37 (1H, m), |
| | methylpyrrolidin- | pyrrolidine | | 3.30 (2H, m), 3.08 (1H, m), |
| | 1-yl)pyridin-2- | [CAS 765-38-8] | | 2.48 (3H, s), 2.16 (1H, m), |
| | amine | | | 2.00 (2H, m), 1.88 (1H, m), |
| | | | | 1.80 (1H, m), |
| | | | | 1.60 (2H, m), 1.20 (3H, d) |

### Example 15

### Synthesis of tert-butyl 4-{2-[(2,2-dimethylpropyl)amino]-6-[(4-methoxy-benzyl)amino]pyridin-4-yl}piperazine-1-carboxylate (Intermediate 17)

Intermediate 12 (30 mg), 2,2′-bis(diphenylphosphino)-1,1′-binaphthyl [CAS 76189-55-4] (3.9 mg), tris(dibenzylideneacetone)dipalladium(0) [CAS 52409-22-0] (1.4 mg), neopentylamine (0.011 ml) and sodium tert-butoxide (21 mg) are placed in a dry flask and sealed. Anhydrous toluene (3 ml) is added. The mixture is degassed with nitrogen, then heated to 80° C. for 18 h. After cooling, the reaction mixture is diluted with EtOAc (15 ml) and washed with saturated sodium hydrogen carbonate (10 ml), saturated brine (10 ml), dried (Na₂SO₄) and evaporated under vacuum to leave an amber gum (36 mg). The crude product is then purified by silica gel column chromatography, with 10% heptane/EtOAc rising to EtOAc as eluent, to provide the title compound as a pale green glass (8 mg, 26%). LCMS 484 [M+H]⁺, RT 2.94 mins (pH 2.5). ¹H NMR 300 MHz (CDCl₃) (δ ppm): 7.27 (2H, d (partly obscured by CHCl₃ peak)), 6.86 (2H, d), 5.70 (1H, m), 5.66 (1H, m), 4.90 (1H, br s), 4.62 (1H, br s), 4.32 (2H, d), 3.80 (3H, s), 3.50 (4H, m), 3.20 (4H, m), 2.94 (2H, d), 1.48 (9H, s), 0.98 (9H, s).

### Example 16

### Synthesis of 6-cyclohex-1-en-1-yl-4-[3-(methylamino)pyrrolidin-1-yl]pyridin-2-amine (Compound 6)

A mixture of intermediate 6 (90 mg), cyclohexen-1-yl boronic acid pinacol ester [CAS 141091-37-4] (80 mg), sodium carbonate (2M aq, 1 ml), chloro(di-2-norbornylphosphino)-(2′-dimethylamino-1,1′-biphenyl-2-yl)palladium (II) [CAS 359803-53-5] (5 mg) in dioxane (5 ml) is heated at 80° C. for 18 h. The mixture is added to water (10 ml) and extracted with EtOAc (10 ml). The organic solvent is dried and evaporated and the crude product dissolved in TFA (5 ml). The solution is heated at reflux for 2 h, then evaporated and the residue purified by preparative HPLC (Method B) to give the title compound as colourless glass (30 mg). LCMS 273 [M+H]⁺, RT (pH 2.5) 1.66 mins. ¹H NMR 300 MHz (MeOD) (δ ppm): 6.35 (1H, m), 6.00 (1H, s), 5.60 (1H, s), 3.55 (1H, m), 3.50 (1H, m), 3.35 (2H, m), 3.15 (1H, m), 2.45 (3H, s), 2.40 (2H, m), 2.20-2.35 (3H, m), 1.90 (1H, m), 1.65-1.80 (4H, m)

Compounds 7 to 15 and 25 are prepared according to the method described in Example 16.

The reagents used and the results obtained are tabulated below.

| | | | M⁺ | ¹H NMR |
|---|---|---|---|---|
| Comp. | | | (M⁺ + 1) | (solvent, |
| No | IUPAC Name | Starting materials | Observed | δ ppm) |
| 7 | 4-[(3- | Intermediate 6 | 337 [M + H⁺] | CDCl₃ 7.98 (2H, d), 7.13 (2H, |
| | methylamino)pyrrolidin- | and | RT | d), 6.33 (1H, s), 5.60 (1H, s), |
| | 1-yl]-6-(4- | 4- | 2.23 min | 4.40 (2H, br s), 3.55 (1H, m), |
| | trifluoromethylphenyl)pyridin- | trifluoromethylphenyl | (pH 5.8) | 3.50 (1H, m), 3.38 (2H, m), |
| | 2- | boronic acid | | 3.13 (1H, m), 2.48 (3H, s), |
| | amine | [CAS 128796-39-4] | | 2.20 (1H, m), 1.87 (1H, m) |
| 8 | 4-[(3- | Intermediate 6 | 353 [M + H⁺] | CDCl₃ 7.87 (2H, d), 7.23 (2H, |
| | methylamino)pyrrolidin- | and | RT | d), 6.25 (1H, s), 5.54 (1H, s), |
| | 1-yl]-6-(4- | 4- | 2.25 min | 4.25 (2H, br s), 3.55 (1H, m), |
| | trifluoromethoxyphenyl)pyridin- | trifluoromethoxybenzene | (pH 5.8) | 3.50 (1H, m), 3.38 (2H, m), |
| | 2- | boronic acid | | 3.13 (1H, m), 2.48 (3H, s), |
| | amine | [CAS 139301-27-2] | | 2.20 (1H, m), 1.87 (1H, m) |
| 9 | 6-(4- | Intermediate 6 | 303 [M + H⁺] | CDCl₃ 7.82 (2H, d), 7.35 (2H, |
| | chlorophenyl)-4- | and | RT | d), 6.30 (1H, s), 5.53 (1H, s), |
| | [(3- | 4-chlorophenyl | 2.11 min | 4.35 (2H, br s), 3.55 (1H, m), |
| | methylamino)pyrrolidin- | boronic acid [CAS | (pH 5.8) | 3.50 (1H, m), 3.38 (2H, m), |
| | 1-yl)pyridin-2- | 1679-18-1] | | 3.13 (1H, m), 2.48 (3H, s), |
| | amine | | | 2.20 (1H, m), 1.87 (1H, m) |
| 10 | 4-[(3- | Intermediate 6 | 283 [M + H⁺] | CDCl₃ 7.72 (1H, s), 7.15 (1H, |
| | methylamino)pyrrolidin- | and | RT | d), 7.30 (1H, t), 7.18 (1H, d), |
| | 1-yl]-6-(3- | 3-methylphenyl | 1.88 min | 6.32 (1H, s), 5.58 (1H, s), |
| | methylphenyl)pyridin- | boronic acid [CAS | (pH 5.8) | 4.67 (2H, br s), 3.55 (1H, m), |
| | 2-amine | 17933-03-8] | | 3.50 (1H, m), 3.38 (2H, m), |
| | | | | 3.13 (1H, m), 2.48 (3H, s), |
| | | | | 2.42 (3H, s), 2.20 (1H, m), |
| | | | | 1.87 (1H, m) |
| 11 | 4-(3- | Intermediate 8 | 259 [M + H⁺] | CDCl₃ 6.60 (1H, m), 6.00 (1H, |
| | aminopyrrolidin-1- | and | RT | s), 5.45 (1H, s), 4.25 (2H, br |
| | yl)-6-cyclohex-1- | 1- | 1.85 min | s), 3.70 (1H, m), |
| | en-1-ylpyridin-2- | cyclohexenyl boronic | (pH 5.8) | 3.42-3.55 (2H, m), 3.32 (1H, m), |
| | amine | acid pinacol ester | | 3.00 (1H, m), 2.40 (2H, m), |
| | | [CAS 141091-37-4] | | 2.15-2.28 (3H, m), 1.50-1.86 (7H, |
| | | | | m) |
| 13 | 6-(3- | Intermediate 9 | 283 [M + H⁺] | CDCl₃ 7.72 (1H, s), 7.68 (1H, |
| | methylphenyl)-4- | and | RT | d), 7.30 (1H, t), 7.20 (1H, d), |
| | (4-methylpiperazin- | 3-methylphenyl | 2.19 min | 6.65 (1H, s), 5.88 (1H, s), |
| | 1-yl)pyridin-2- | boronic acid [CAS | (pH 5.8) | 4.39 (2H, br s), 3.38 (4H, m), |
| | amine | 17933-03-8] | | 2.60 (4H, m), 2.44 (3H, s), |
| | | | | 2.40 (3H, s) |
| 14 | 6-(4-chlorophenyl)- | Intermediate 9 | 303 [M + H⁺] | DMSO 8.00 (2H, d), 7.41 (2H, |
| | 4-(4- | and | RT | d), 6.73 (1H, s), 5.85 (1H, s), |
| | methylpiperazin-1- | 4-chlorophenyl | 2.23 min | 5.60 (2H, br s), 3.32 (4H, m), |
| | yl)pyridin-2-amine | boronic acid [CAS | (pH 5.8) | 2.43 (4H, m), 2.23 (3H, s) |
| | | 1679-18-1] | | |
| 15 | 6-(4- | Intermediate 9 | 287 [M + H⁺] | CDCl₃ 6.58 (1H, m), |
| | methylcyclohex-1- | and | RT | 6.29 (1H, s), 5.78 (1H, s), |
| | en-1-yl)-4-(4- | 4- | 2.30 min | 4.23 (2H, br s), 3.30 (4H, m), |
| | methylpiperazin-1- | methylcyclohexen- | (pH 5.8) | 2.50 (4H, m), 2.40 (1H, m), |
| | yl)pyridin-2-amine | 1-yl boronic acid | | 2.32 (3H, s), 2.29 (1H, m), |
| | | [CAS 850567-92-9] | | 1.68-1.87 (5 H, m), 1.33 (1H, m), |
| | | | | 1.00 (3H, d) |
| 25 | 6-(4- | Intermediate 12, | 289 [M + H⁺] | (CDCl₃) 7.80 (2H, d), |
| | Chlorophenyl)-4- | and | RT1.92 min | 7.35 (2H, d), 6.58 (1H, m), |
| | piperazin-1- | 4-chlorophenyl- | (pH | 5.83 (1H, m), 4.42 (2H, br s), |
| | ylpyridin-2-amine | boronic acid | 5.8 | 3.30 (4H, m), 3.00 (4H, m), |
| | | [CAS 1679-18-1] | | 1.87 (1H, br s) |

### Example 16

### Synthesis of 4-[(3R)-3-aminopyrrolidin-1-yl]-6-(4-chlorophenyl)pyridin-2-amine (Compound 12)

A solution of Intermediate 16 (15 mg), 4-chlorophenylboronic acid [CAS 1679-18-1] (20 mg) and sodium carbonate (2M aq., 0.3 ml) in DME (1 ml) is degassed by passage of nitrogen, then tetrakis(triphenylphosphine)palladium(0) [CAS14221-01-3] (2 mg) is added and the mixture heated in a microwave reactor at 150° C. for 1 h. The vessel is opened and fresh catalyst (3 mg) added, then the mixture is heated for 1 h at 150° C. The solution is added to water (10 ml), extracted with EtOAc (10 ml) and the organic solvent dried and evaporated. The crude residue is dissolved in TFA (3 ml) and heated at reflux for 1 h, then cooled and evaporated and the residue purified by silica gel column chromatography, with 10% MeOH/DCM 1% NH₄OH as eluent, to give the title compound as colourless solid (4.3 mg, 43%). LCMS 289/291 [M+H]⁺, RT 2.12 mins (pH 5.8). ¹H NMR 300 MHz (CDCl₃) (δ ppm): 7.82 (2H, d), 7.35 (2H, d), 6.28 (1H, m), 5.55 (1H, m), 4.52 (2H, br s), 3.73 (1H, m), 3.50-3.60 (2H, m), 3.37 (1H, m), 3.08 (1H, m), 2.12 (1H, m), 1.78 (1H, m), 1.70 (2H, br s).

### Example 17

### Synthesis of 6-(2,4-difluorophenyl)-4-[3-(methylamino)pyrrolidin-1-yl]pyridin-2-amine (compound 18)

A solution of Intermediate 6 (100 mg) and 2,4-difluorophenylboronic acid (100 mg) in a mixture of dioxane (2 ml) and sodium carbonate (2M aq, 1 ml) is degassed with a stream of nitrogen gas, then chloro(di-2-norbornylphosphino)-(2′-dimethylamino-1,1′-biphenyl-2-yl)palladium(II) [CAS 359803-53-5] (5 mg) is added and the mixture heated under microwave irradiation at 140° C. for 1 h. The mixture is added to water (10 ml) and extracted with EtOAc (2×10 ml), the organic solvent dried and evaporated and the residue dissolved in TFA (5 ml) and heated at reflux for 1 h. The solution is evaporated in vacuo and the residue purified by preparative HPLC (method B). The collected fractions are evaporated in vacuo, dissolved in water, basified with NaOH (2M aq) and extracted with EtOAc. The organic solvent is dried and evaporated to give the title compound as white solid (25 mg, 35%). LCMS 305 [M+H]⁺, RT 1.63 mins (pH 5.8). ¹H NMR 300 MHz (CDCl₃) (δ ppm): 7.75 (1H, m), 6.83-7.00 (2H, m), 6.24 (1H, m), 5.75 (2H, br s), 5.53 (1H, m), 3.55 (1H, m), 3.30-3.43 (3H, m), 3.20 (1H, m), 3.15 (1H, br s), 2.50 (3H, s), 2.23 (1H, m), 1.95 (1H, m).

Compounds 19 to 24 are prepared according to the method described in Example 17.

The reagents used and the results obtained are tabulated below.

| | | | | M⁺ | ¹H NMR |
|---|---|---|---|---|---|
| Comp. | | | | (M⁺ + 1) | (solvent, |
| No | IUPAC Name | Starting materials | Salt | Observed | δ ppm) |
| 19 | 6-[(1E)-3,3- | Intermediate 6, | | 275 [M + H]⁺ | (CDCl₃) 6.75 (1H, d), |
| | Dimethylbut-1- | and 3,3-dimethyl- | | RT 2.15 mins | 6.18 (1H, d), |
| | en-1-yl]-4-[3- | 1-butenylboronic | | (pH | 5.93 (1H, m), 5.43 (1H, |
| | (methylamino)pyrrolidin- | acid [CAS 86595- | | 5.8) | m), 4.35 (2H, br s), |
| | 1- | 37-1] | | | 3.50 (1H, m), |
| | yl]pyridin-2- | | | | 3.28-3.43 (3H, m), |
| | amine | | | | 3.10 (1H, m), 2.49 (3H, s), |
| | | | | | 2.18 (1H, m), |
| | | | | | 1.95 (1H, br s), 1.80 (1H, |
| | | | | | m), 1.15 (9H, s) |
| 20 | 6-[(E)-2- | Intermediate 6, | Acetate | 259 [M + H]⁺ | (CD₃OD) 6.35 (1H, |
| | Cyclopropylvinyl]- | and 2- | | RT 1.53 mins | d), 6.22 (1H, m), |
| | 4-[3- | cyclopropylvinylboronic | | (pH | 6.12 (1H, dd), 5.63 (1H, |
| | (methylamino)pyrrolidin- | acid [CAS | | 5.8) | m), 3.55-3.70 (2H, |
| | 1- | 903510-64-5] | | | m), 3.42-3.55 (2H, |
| | yl]pyridin-2- | | | | m), 3.30 (1H, m), |
| | amine | | | | 2.48 (3H, s), |
| | | | | | 2.30 (1H, m), 2.00 (1H, |
| | | | | | m), 1.93 (3H, s), |
| | | | | | 1.68 (1H, m), 1.00 (2H, |
| | | | | | m), 0.68 (2H, m) |
| 21 | 6-(1- | Intermediate 6, | | 325 [M + H]⁺ | (CDCl₃) |
| | Benzothien-5- | and thiophene-5- | | RT 2.27 mins | 7.74-7.83 (3H, m), |
| | yl)-4-[3- | boronic acid | | (pH | 7.26-7.37 (2H, m), 6.45 (1H, |
| | (methylamino)pyrrolidin- | [CAS 845872-49- | | 5.8) | m), 5.55 (1H, m), |
| | 1- | 3] | | | 4.35 (2H, br s), |
| | yl]pyridin-2- | | | | 3.55 (1H, m), 3.48 (1H, |
| | amine | | | | m), 3.38 (2H, m), |
| | | | | | 3.17 (1H, m), |
| | | | | | 2.52 (3H, s), 2.22 (1H, m), |
| | | | | | 1.87 (1H, m), |
| | | | | | 1.75 (1H, br s) |
| 23 | 6-(4-tert- | Intermediate 6, | | 329 [M + H]⁺ | (CD₃OD) 6.37 (1H, |
| | Butylcyclohex-1- | and 4-tert- | | RT 2.27 mins | m), 6.00 (1H, m), |
| | en-1-yl)-4-[3- | butylcyclohexen- | | (pH | 5.60 (1H, m), |
| | (methylamino)pyrrolidin- | 1-ylboronic acid | | 5.8) | 3.55 (1H, m), 3.50 (1H, |
| | 1- | [CAS] | | | m), 3.35 (2H, m), |
| | yl]pyridin-2- | | | | 3.12 (1H, m), |
| | amine | | | | 2.55 (1H, m), 2.43 (3H, s), |
| | | | | | 2.28-2.40 (2H, m), |
| | | | | | 2.23 (1H, m), |
| | | | | | 2.07 (2H, m), 1.93 (1H, |
| | | | | | m), 1.25-1.45 2H, m), |
| | | | | | 0.93 (9H, s) |
| 24 | 6-(3,4- | Intermediate 6, | | 337 [M + H]⁺ | (CDCl₃) 8.00 (1H, d), |
| | Dichlorophenyl)- | and 3,4- | | RT 2.24 mins | 7.72 (1H, dd), |
| | 4-[3- | dichlorophenylboronic | | (pH | 7.45 (1H, d), 6.26 (1H, m), |
| | (methylamino)pyrrolidin- | acid [CAS | | 5.8) | 5.52 (1H, m), |
| | 1- | 151169-74-3] | | | 4.43 (2H, br s), 3.53 (1H, |
| | yl]pyridin-2- | | | | m), 3.48 (1H, m), |
| | amine | | | | 3.33-3.42 (2H, m), |
| | | | | | 3.15 (1H, m), |
| | | | | | 2.50 (3H, s), 2.23 (1H, m), |
| | | | | | 1.88 (1H, m) |

### Example 18

### Synthesis of 4-[3-(methylamino)azetidin-1-yl]-6-phenylpyridin-2-amine, bisacetate salt (Compound 26)

A mixture of Intermediate 4 (200 mg), 4-chlorophenylboronic acid [CAS 1679-18-1] (200 mg) and sodium carbonate (2M aq., 1 ml) in DME (5 ml) is degassed by passage of nitrogen, then chloro(di-2-norbornylphosphino)-(2′-dimethylamino-1,1′-biphenyl-2-yl)palladium (II) [CAS 359803-53-5] (5 mg) is added. The mixture is heated at 80° C. for 18 h then cooled, added to water (20 ml) and extracted with EtOAc (2×20 ml). The organic solvent is dried and evaporated and the crude product is dissolved in THF (20 ml) and heated with lithium aluminium hydride (100 mg) for 18 h. A further portion of lithium aluminium hydride is added and heating continued for 24 h, then water (0.2 ml) is added, followed by sodium hydroxide (1 M aq, 0.2 ml) and water (0.6 ml). The mixture is stirred for 30 mins then filtered through Celite and the filtrate evaporated in vacuo. The residue is purified by preparative HPLC (method B) to give the title compound as colourless solid (12 mg, 11%). LCMS 255 [M+H]⁺, RT 1.66 mins (pH 5.8). ¹H NMR 300 MHz (CD₃OD) (δ ppm): 7.73 (2H, m), 7.55 (3H, m), 6.29 (1H, m), 5.58 (1H, m), 4.35 (2H, m), 3.97 (2H, m), 3.84 (1H, m), 2.43 (3H, s), 1.93 (6H, s (2×AcOH)).

### Example 19

### Synthesis of 6-(2-adamantyl)-4-(4-methylpiperazin-1-ylpyridyl-2-amine (Compound 16)

A solution of Intermediate 10 (20 mg) in TFA (5 ml) is heated at reflux for 1 h, then evaporated and the residue purified by chromatography (6% MeOH/DCM 1% NH₄OH) to give the title compound as beige solid (9.6 mg). LCMS 327 [M+H]⁺, RT (pH 5.8) 2.48 mins. ¹H NMR 300 MHz (CDCl₃) (δ ppm): 6.29 (1H, s), 5.76 (1H, s), 4.25 (2H, br s), 3.30 (4H, m), 2.83 (1H, m), 2.50-2.63 (6H, m), 2.35 (3H, s), 1.50-2.00 (12H, m).

### Example 20

### Synthesis of N-(2,2-dimethylpropyl)-4-piperazin-1-ylpyridine-2,6-diamine (Compound 38)

Compound 38 is prepared from Intermediate 17 (20 mg), in a similar manner to the method described for Compound 16, to give the title compound as an off-white solid (3 mg, 28%). LCMS 264 [M+H]⁺, RT 1.60 mins (pH 5.8). ¹H NMR 300 MHz (CD₃OD) (δ ppm): 5.47 (1H, s), 5.39 (1H, s), 3.28 (4H, m), 2.97 (2H, s), 2.92 (4H, m), 1.00 (9H, s).

### Example 21

### Synthesis of 6-adamantan-2-yl-4-[3-(methylamino)pyrrolidin-1-yl]pyridin-2-amine (Compound 22)

A solution of Intermediate 6 (100 mg), 2-adamantyl zinc bromide [CAS 171860-65-4] (0.5 M in THF, 0.5 ml) and Pd(dppf)Cl₂ [CAS 72287-26-4] (10 mg) is heated at reflux overnight. The reaction mixture is cooled and evaporated and the residue dissolved in TFA (10 ml) and heated at reflux for 1 h. The solution is evaporated in vacuo and the residue purified by preparative HPLC (method B) to give the title compound as colourless solid (10 mg, 10%). LCMS 327 [M+H]⁺, RT 2.07 mins (pH 5.8). ¹H NMR 300 MHz (CD₃OD) (δ ppm): 6.03 (1H, m), 5.58 (1H, m), 3.55 (1H, m), 3.52 (1H, m), 3.35 (2H, m), 3.17 (1H, m), 2.82 (1H, m), 2.47 (2H, m), 2.45 (3H, s), 2.25 (1H, m), 1.88-2.10 (9H, m), 1.82 (2H, m), 1.63 (2H, m).

Compounds 37 to 39 are prepared according to the method described in Example 21.

The reagents used and the results obtained are tabulated below.

| | | | M⁺ | ¹H NMR |
|---|---|---|---|---|
| Comp. | | | (M⁺ + 1) | (solvent, |
| No | IUPAC Name | Starting materials | Observed | δ ppm) |
| 37 | 6-Adamantan-2-yl- | Int 12, and 2- | 313 [M + H]⁺ | (CDCl₃) 6.23 (1H, m), |
| | 4-piperazin-1- | adamantyl zinc | RT 2.08 mins | 5.71 (1H, m), 4.86 (2H, |
| | ylpyridin-2-amine | bromide [CAS | (pH | br s), 3.28 (4H, m), |
| | | 171860-65-4] | 5.8) | 3.10 (1H, br s), 2.95 (4H, m), |
| | | | | 2.88 (1H, m), 2.50 (2H, |
| | | | | m), 1.85-2.10 (8H, m), |
| | | | | 1.80 (2H, m), 1.57 (2H, |
| | | | | m) |
| 39 | 6-Cyclohexyl-4-[3- | Intermediate 6, | 275 [M + H]⁺ | (CDCl₃) 5.82 (1H, m), |
| | (methylamino)pyrrolidin- | and | RT 1.71 mins | 5.45 (1H, m), 4.20 (2H, |
| | 1-yl]pyridin-2- | cyclohexylzinc | (pH | br s), 3.30-3.53 (4H, |
| | amine | bromide [CAS | 5.8) | m), 3.10 (1H, m), |
| | | 7565-57-3] | | 2.50 (3H, s), 2.42 (1H, m), |
| | | | | 2.20 (1H, m), |
| | | | | 1.60-2.00 (6H, m), 1.20-1.55 (6H, |
| | | | | m) |

### BIOLOGICAL EXAMPLES

### Example 22

### Human H₄R³Histamine Binding Assay

Cf. The Journal of Pharmacology and Experimental Therapeutics 2001, 299(1); 121-130.

³Histamine dihydrochloride (Amersham) binding to the human H₄ receptor is determined using CHO-hH₄R membranes (350 ug/ml; Euroscreen), SPA beads (GE Healthcare; 15 mg/ml) and histamine (20 μM) in assay buffer [Tris HCl (50 mM), EDTA (5 mM, pH 7.4), 0.1% fatty acid free BSA]. The test compounds (0.5% DMSO final) are incubated with the assay mix in 96-well Optiplates (Perkin Elmer) for 15 mins at room temperature prior to addition of ³H-histamine solution (10 nM); the final assay volume is 200 μl per well. The plates are sealed and incubated for 16 h at room temperature prior detection of membrane bound radioligand on Topcount (Perkin Elmer). Unless noted, all reagents are purchased from Sigma. Affinity (pKᵢ) measurements are determined by assessing the concentration of compound necessary to displace 50% of the specifically bound ³H-histamine.
The compounds of the invention including are tested in this assay; their Kᵢ/EC₅₀ measurements are of less than 10 μM. The preferred compounds of the invention give Kᵢ/EC₅₀ measurements less than 1 μM. Most preferred compounds have activities less than 100 nM.
Compound 8, 4-[(3-methylamino)pyrrolidin-1-yl]-6-(4-trifluoromethoxyphenyl)pyridin-2-amine, gives a Kᵢ/EC₅₀ between 200 and 450 nM.

### Example 23

### Human H₄ GTPγS³⁵ Assay

Cf. The Journal of Pharmacology and Experimental Therapeutics 2000, 296(3); 1058-1066.

GTPγS³⁵ (Amersham) binding is determined using CHO-hH₄R membranes (Euroscreen; 50 μg/ml), SPA beads (GE Healthcare; 10 mg/ml), GDP (15 μM) and saponin (30 μg/ml) in assay buffer [20 mM Hepes, 100 mM NaCl, 10 mM MgCl, 1 mM EDTA (pH 7.4), 0.1% BSA) in 96-well Optiplates (Perkin Elmer). Test compounds (0.5% DMSO final) are added and plates are incubated for 1 h at room temperature. GTPγS³⁵ (300 pM) is added (final assay volume 200 μl/well) and plates are incubated for a further 90 mins at room temperature prior to centrifugation of plates and detection using Topcount (Perkin Elmer). Unless noted, all reagents are purchased from Sigma. Affinity/efficacy measurements (pKᵢ/pEC₅₀) are determined by assessing the concentration of compound necessary to inhibit 50% of the functional response to a fixed concentration of histamine (GTPγS³⁵ binding), or the concentration of compound to cause a 50% increase in GTPγS³⁵ binding. The compounds of the invention are tested in this assay their Kᵢ/EC₅₀ measurements are of less than 10 μM. The preferred compounds of the invention give Kᵢ/EC₅₀ measurements less than 1 μM. Most preferred compounds have activities less than 100 nM.
Compound 8, 4-[(3-methylamino)pyrrolidin-1-yl]-6-(4-trifluoromethoxyphenyl)pyridin-2-amine, gives a Kᵢ/EC₅₀ between 75 and 250 nM.

1. A compound having formula I or pharmaceutically acceptable salts thereof or stereoisomeric forms thereof, and the geometrical isomers, enantiomers, diastereoisomers, and pharmaceutically acceptable salts thereof 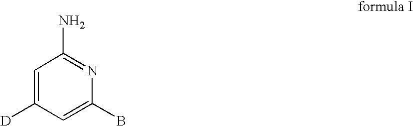
* represents the point of attachment to the rest of the molecule
wherein:
B is aryl optionally substituted with a substituent selected from the group consisting of C₁₋₆ alkyl, halogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl;
or B is a heteroaryl ring optionally substituted with a substituent selected from the group consisting of C₁₋₆ alkyl, halogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl;
or is C₁₋₆ alkyl optionally substituted with a substituent selected from the group consisting of aryl, heteroaryl, 3-10 member cycloalkyl, 3-10 member heterocycloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine;
or B is C₂₋₆ alkenyl optionally substituted with a substituent selected from the group consisting of C₁₋₆ alkyl, aryl, heteroaryl, 3-10 member cycloalkyl, 3-10 member heterocycloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine;
or B is C₃₋₁₀ cycloalkyl optionally substituted by C₁₋₆ alkyl;
or B is C₃₋₁₀ cycloalkenyl optionally substituted by C₁₋₆ alkyl;
or B is a group of formula II 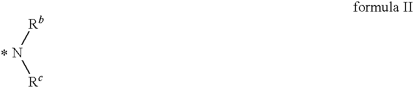
wherein
R^{b} is hydrogen or C₁₋₆ alkyl;
R^{c} is C₃₋₁₀ cycloalkyl optionally substituted by C₁₋₃ alkyl;
or R^{b} and R^{c} can form together with the nitrogen, a 3-10 member heterocycloalkyl or a heteroaryl optionally substituted by C₁₋₆ alkyl;
D is a group of formula III 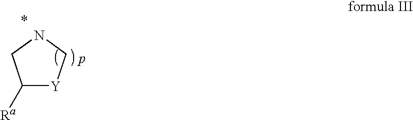
wherein
R^{a} is hydrogen or unsubstituted C₁₋₃ alkyl;
p is 1 or 2 or 3;
Y is a group of formula IV 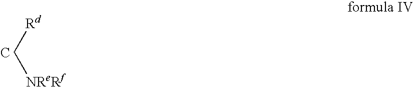
wherein
R^{d} is hydrogen or unsubstituted C₁₋₃ alkyl;
R^{e} is hydrogen or unsubstituted C₁₋₃ alkyl;
R^{f} is hydrogen or unsubstituted C₁₋₃ alkyl;
or Y is a group of formula V
N—R^{g} formula V
wherein
R^{g} is hydrogen or unsubstituted C₁₋₃ alkyl;
or D is a group of formula VI 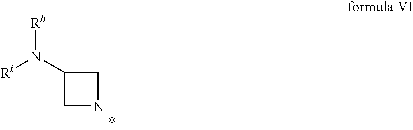
wherein
R^{h} is hydrogen or unsubstituted C₁₋₃ alkyl;
Rⁱ is hydrogen or unsubstituted C₁₋₃ alkyl;
or D is a group of formula VII 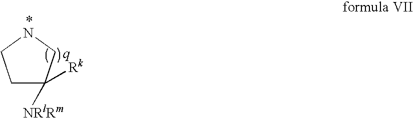
wherein
q is 1 or 2 or 3;
R^{k} is hydrogen or unsubstituted C₁₋₃ alkyl;
R^{l} is hydrogen or unsubstituted C₁₋₃ alkyl;
R^{m} is hydrogen or unsubstituted C₁₋₃ alkyl;
or D is group of formula VIII 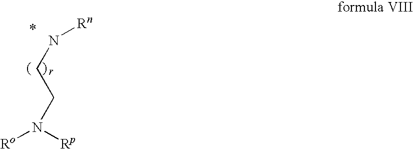
wherein
r is 1 or 2;
Rⁿ is hydrogen or unsubstituted C₁₋₃ alkyl;
R^{o} is hydrogen or unsubstituted C₁₋₃ alkyl;
R^{p} is hydrogen or unsubstituted C₁₋₃ alkyl;
or D is a group of formula IX 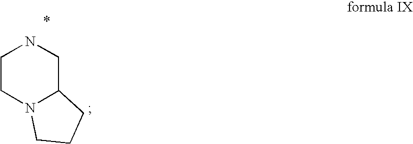
or D is a group of formula X 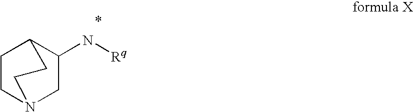
wherein
R^{q} is hydrogen or unsubstituted C₁₋₃ alkyl;
or D is a group of formula XI 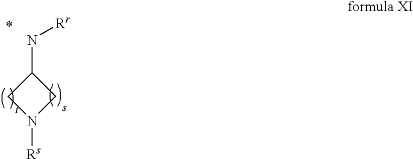
wherein
R^{r} is hydrogen or unsubstituted C₁₋₃ alkyl;
R^{s} is hydrogen or unsubstituted C₁₋₃ alkyl;
s is 1 or 2
t is 1 or 2;
or D is a group of formula XII 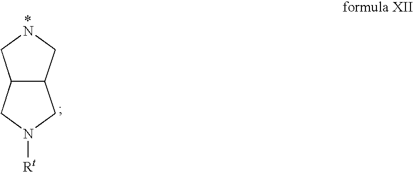
wherein
R^{t} is hydrogen or unsubstituted C₁₋₃ alkyl.

2. The compound according to claim 1 wherein B is aryl optionally substituted with a substituent selected from the group consisting of C₁₋₆ alkyl, halogen, hydrogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl; and D is a group of formula III wherein R^{a} is C₁₋₃ alkyl, hydrogen; and p is 1 or 2 or 3; and Y is a group of formula IV wherein R^{d} is hydrogen or C₁₋₃ alkyl; R^{e} is hydrogen or C₁₋₃ alkyl; R^{f} is hydrogen or C₁₋₃ alkyl.

3. The compound according to claim 1 wherein B is C₃₋₁₀ cycloalkyl optionally substituted by C₁₋₆ alkyl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

4. The compound according to claim 1 wherein B is C₃₋₁₀ cycloalkenyl optionally substituted by C₁₋₆ alkyl; and D is a group of formula III wherein R^{a} is C₁₋₃ alkyl, hydrogen; and p is for 2 or 3; and Y is a group of formula IV wherein R^{d} is hydrogen or C₁₋₃ alkyl; R^{e} is hydrogen or C₁₋₃ alkyl; R^{f} is hydrogen or C₁₋₃ alkyl.

5. The compound according to claim 1 wherein B is C₃₋₁₀ cycloalkenyl optionally substituted by C₁₋₆ alkyl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

6. The compound according to claim 1 wherein aryl optionally substituted with a substituent selected from the group consisting of C₁₋₆ alkyl, halogen, hydrogen, C₁₋₃ haloalkyl, hydroxyl, C₁₋₃ alkoxy, C₁₋₃ haloalkoxy, carboxylic acid, ester, C₁₋₃ alkylsulfonyl, amide, sulfonamide, C₁₋₃ dialkylamine, 3-10 member heterocycloalkyl, heteroaryl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

7. The compound according to claim 1 wherein B is a group of formula II wherein R^{b} is hydrogen, C₁₋₆ alkyl; R^{c} is C₃₋₁₀ cycloalkyl optionally substituted by C₁₋₃ alkyl; and D is a group of formula III wherein R^{a} is C₁₋₃ alkyl, hydrogen; and p is 1 or 2 or 3; and Y is a group of formula V wherein R^{g} is hydrogen or C₁₋₃ alkyl.

8. The compound according to claim 1 wherein B is a group of formula II wherein R^{b} and R^{c} can form together with the nitrogen, a 3-10 member heterocycloalkyl optionally substituted by C₁₋₆ alkyl; and D is a group of formula III wherein R^{a} is hydrogen; and p is 1; and Y is a group of formula IV wherein Rd is hydrogen; R^{e} is hydrogen; and R^{f} is C₁₋₃ alkyl.

9. The compound according to claim 1 wherein B is a group of formula II wherein R^{b} and R^{c} can form together with the Nitrogen atom a 3-10 member heterocycloalkyl or heteroaryl optionally substituted by C₁₋₆ alkyl; and D is a group of formula III wherein R^{a} is C₁₋₃ alkyl, hydrogen; and p is 1 or 2 or 3; and Y is a group of formula IV wherein R^{d} is hydrogen, C₁₋₃ alkyl; R^{e} is hydrogen, C₁₋₃ alkyl; R^{f} is hydrogen, C₁₋₃ alkyl.

10. The compound according to claim 1 wherein B is a group of formula II wherein R^{b} and R^{c} can form together with the nitrogen, a 3-10 member heterocycloalkyl optionally substituted by C₁₋₆ alkyl; and D is a group of formula III wherein R^{a} is hydrogen; and p is 1; and Y is a group of formula IV wherein Rd is hydrogen; R^{e} is hydrogen; and R^{f} is C₁₋₃ alkyl.

11. The compound according to claim 1 wherein B is a group of formula II wherein R^{b} is hydrogen; R^{c} is C₃₋₁₀ cycloalkyl; and D is a group of formula III wherein R^{a} is hydrogen; and p is 2; and Y is a group of formula V wherein R^{g} is C₁₋₃ alkyl.

12. The compound according to claim 1 wherein B is an aryl optionally substituted by C₁₋₆ alkyl; and D is a group of formula III wherein R^{a} is hydrogen; and p is 2; and Y is a group of formula V wherein R^{g} is C₁₋₃ alkyl.

13. The compound according to claim 1 wherein B is aryl optionally substituted by halogen; and D is a group of formula III wherein R^{a} is hydrogen; and p is 1; and Y is a group of formula IV wherein R^{d} is hydrogen; and R^{e} is hydrogen; and R^{f} is hydrogen or C₁₋₃ alkyl.

14. The compound according to claim 1 wherein B is C₃₋₁₀ cycloalkenyl optionally by C₁₋₆ alkyl group; and D is a group of formula III wherein R^{a} is hydrogen; and p is 1; and Y is a group of formula IV wherein R^{d} is hydrogen; and R^{e} is hydrogen; and R^{f} is C₁₋₃ alkyl.

15. The compound according to claim 1 selected from the group consisting of:
6-cyclohex-1-en-1-yl-4-[3-(methylamino)pyrrolidin-1-yl]pyridin-2-amine-;
6-(4-chlorophenyl)-4-(4-methylpiperazin-1-yl)pyridin-2-amine;
6-(4-methylcyclohex-1-en-1-yl)-4-(4-methylpiperazin-1-yl)pyridin-2-amine;
6-adamantan-2-yl-4-(4-methylpiperazin-1-yl)pyridin-2-amine;
4-(3-aminopyrrolidin-1-yl)-6-cyclohex-1-en-1-ylpyridin-2-amine;
6-(3-methylphenyl)-4-(4-methylpiperazin-1-yl)pyridin-2-amine;
4-[(3R)-3-aminopyrrolidin-1-yl]-6-(4-chlorophenyl)pyridin-2-amine;
4-(4-methylpiperazin-1-yl)-6-(4-methylpiperidin-1-yl)pyridin-2-amine;
N-cycloheptyl-4-(4-methylpiperazin-1-yl)pyridine-2,6-diamine;
4-[3-(methylamino)pyrrolidin-1-yl]-6-(2-methylpyrrolidin-1-yl)pyridin-2-amine;
4-(4-methylpiperazin-1-yl)-6-(2-methylpyrrolidin-1-yl)pyridin-2-amine;
4-[3-(methylamino)pyrrolidin-1-yl]-6-(4-methylpiperidin-1-yl)pyridin-2-amine;
4-[(3-methylamino)pyrrolidin-1-yl]-6-(4-trifluoromethylphenyl)pyridin-2-amine;
4-[(3-methylamino)pyrrolidin-1-yl]-6-(4-trifluoromethoxyphenyl)pyridin-2-amine;
6-(4-chlorophenyl)-4-[(3-methylamino)pyrrolidin-1-yl)pyridin-2-amine;
4-[(3-methylamino)pyrrolidin-1-yl]-6-(3-methylphenyl)pyridin-2-amine.

16. The compound according to claim 1 selected from the group consisting of:
N-cycloheptyl-4-(4-methylpiperazin-1-yl)pyridine-2,6-diamine;
4-[3-(methylamino)pyrrolidin-1-yl]-6-(2-methylpyrrolidin-1-yl)pyridin-2-amine;
4-[(3-methylamino)pyrrolidin-1-yl]-6-(3-methylphenyl)pyridin-2-amine.
6-cyclohex-1-en-1-yl-4-[3-(methylamino)pyrrolidin-1-yl]pyridin-2-amine-;
6-(4-chlorophenyl)-4-[(3-methylamino)pyrrolidin-1-yl)pyridin-2-amine.

17. Synthesis intermediates selected from a group consisting of:
2,6-difluoro-4-(4-methylpiperazinyl)pyridine;
2-fluoro-4-(4-methylpiperazinyl)-6-(4-methoxybenzylamino)pyridine;
2,6-difluoro-4-((3-N-methyl-N-tertbutoxycarbonylamino)pyrrolidino)-pyridine;
2-(4-methoxybenzylamino)-4-((3-N-methyl-N-tertbutoxycarbonylamino)pyrrolidino)-6-fluoropyridine;
2,6-dibromo-4-((3-tertbutoxycarbonylamino)pyrrolidino)-pyridine;
2-bromo-4-((3-N-methyl,N-tertbutoxycarbonylamino)pyrrolidino)-6-(4-methoxybenzylamino-pyridine;
2,6-dichloro-4-((N-tertbutoxycarbonylamino)pyrrolidino)pyridine;
2-chloro-6-(4-methoxybenzylamino)-4-((N-tertbutoxycarbonylamino)pyrrolidino)pyridine;
2-chloro-4-(4-methylpiperazin-1-yl)-6-(4-methoxybenzylamino)pyridine;
2-(2-adamantyl)-4-(4-methylpiperazin-1-yl)-6-(4-methoxybenzylamino)pyridine.

18. A pharmaceutical composition comprising as active ingredient a therapeutically effective amount of a compound according to claim 1 and a pharmaceutically acceptable adjuvant, diluent or carrier.

19. (canceled)

20. A method of treating an H₄ dependent disease in a subject in need thereof comprising administering a compound or a pharmaceutical composition comprising a therapeutically effective amount of a compound according to claim 1.

21. The method of claim 21 wherein the H₄ dependent disease is adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; thromboembolic diseases, atherosclerosis, myocardial infarction, angina pectoris, (unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; or lymphatic diseases.

22. The method of claim 21 wherein the H₄ dependent disease is adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin or diseases of the gastrointestinal tract including inflammatory bowel disease, Crohn's disease, ulcerative colitis, or autoimmune diseases including rheumatoid arthritis, or multiple sclerosis.

23.-24. (canceled)
